(19) United States
(12) Patent Application Publication
(10) Pub. No.: US 2002/0034486 A1
(43) Pub. Date: Mar. 21, 2002

(54) Leave-in hair cosmetic compositions for enhancing volume

(76) Inventors: Sanjeev Midha, Mason, OH (US); Shari Renee Thomson, Cincinnati, OH (US); Michael Albert Snyder, Kobe (JP)

Correspondence Address: DINSMORE & SHOHL, LLP, 1900 CHEMED CENTER, 255 EAST FIFTH STREET, CINCINNATI, OH, 45202, US

(21) Appl. No.: 09/822,704

(22) Filed: Mar. 30, 2001

Related U.S. Application Data
(63) Continuation-in-part of application No. PCT/US00/08760, filed on Mar. 31, 2000.
(60) Provisional application No. 60/231,152, filed on Sep. 8, 2000.
(60) Provisional application No. 60/261,384, filed on Jan. 12, 2001.

(51) Int. Cl. A61K 7/09

(52) U.S. Cl. 424/70.2

(57) ABSTRACT

Leave-in hair cosmetic compositions for enhancing hair volume comprise non-spherical microparticles exhibiting a mean particle size of less than about 100 μm in its longest dimension, a water-soluble or water-swellable polymer, and an aqueous carrier such that the combination of the polymer and the microparticles results in a film-forming network. Also disclosed are methods for enhancing hair volume, and more particularly for enhancing hair volume with leave-in aqueous cosmetic compositions which contain non-spherical microparticles of less than 100 μm in its longest dimension, a water-soluble or water-swellable polymer and an aqueous carrier. The disclosed compositions provide improved hair volume, body, bounce, fullness, springiness, and texture in addition to providing good hair conditioning and styling benefits. Fluid-encapsulated, flexible microspheres which exhibit a mean particle size of less than about 300 μm in diameter may also be included in the compositions.

### RELATED APPLICATIONS

This application claims priority under 35 U.S.C. §120 of International Application PCT/00/8760 filed Mar. 31, 2000 and under 35 U.S.C. §119 of U.S. patent applications Ser. Nos. 60/231,152 filed Sep. 8, 2000 and 60/261,384 filed Jan. 12, 2001.

### TECHNICAL FIELD

The present invention relates to leave-in hair cosmetic compositions comprising non-spherical microparticles and a water-soluble or water-swellable polymer for enhancement of the volume of the hair. The compositions may optionally further comprise fluid-encapsulated, flexible microspheres.

### BACKGROUND OF THE INVENTION

It is well known that there exists a distinct group of people who have fine, thin hair. In order to achieve good hair volume, which is the visible bulkiness of hair, these people desire more body and fullness from their hair. There are many factors that influence hair body and fullness: hair diameter, hair fiber-to-fiber interactions, natural configuration (kinky, straight, wavy), bending stiffness, hair density (# per cm²), and hair length. People use styling products to alter fiber-to-fiber interactions and lock their created styles in place. Many people change the nature of their hair substrate by perming, hair straightening, back combing, and pressing. In the past, technologists have tried to increase the diameter of the hair, but attempts resulted in insignificant gains or in severe hair damage. People want to have good hair feel and people with damaged hair desire to condition their hair. Hair conditioners, both leave-in and rinse-off, improve the wet and dry combing of hair and the feel of the hair. Typical ingredients used in conditioner form a thin coating of low friction polymers or polymer-surfactant complexes on hair fibers. This results in good combing benefits because of reduced surface friction of hair fibers. However, reduced friction has a negative impact on achieving and maintaining the desired hair volume. This happens particularly for people who have fine and thin hair. So there exists a need to have conditioning products that also increase or enhance hair volume.

Styling products are used by consumers to create and maintain their hair style. This is particularly true for the hair styling gels and mousse products. During their wet state on hair, gel and mousse products help in increasing the grab of hair fibers by a hair comb or brush and thereby help in the creation of a hair style. On drying, polymeric bonds are formed in between and on the surface of hair fibers. These bonds help in holding and maintaining the created hair volume and style. Polymeric bonds are broken when the hair is combed or brushed. Broken bonds have jagged edges which enhance inter fiber friction and hence help in maintaining the hair volume and style. It has been discovered that the addition of non-spherical microparticles and fluid-encapsulated, flexible microspheres in conditioning and styling compositions increase the hair volume and body.

It is an objective of this invention to provide leave-in hair cosmetic compositions utilizing non-spherical microparticles and water-soluble or water-swellable polymers that provide improved hair volume, body, bounce, fullness, springiness, and texture in addition to providing good hair conditioning and styling benefits.

### SUMMARY OF THE INVENTION

The present invention is directed to leave-in hair cosmetic compositions, comprising non-spherical microparticles exhibiting a mean particle size of less than about 100 μm in its longest dimensions, a water-soluble or water-swellable polymer, and an aqueous carrier, wherein the combination of the polymer and the microparticles results in a film-forming network. The compositions may further comprise fluid-encapsulated, flexible microspheres exhibiting a mean particle size of less than about 300 μm in diameter, which microspheres, in combination with the polymer and the microparticles, contribute to the film-forming network.

It has been discovered that combination of non-spherical, low density microparticles with the water-soluble or water-swellable polymer, delivered from an aqueous carrier results in a solid continuous or semi-continuous film network. This unique combination results in reduced film density, and a textured or a mated surface which increases hair-to-hair interactions. Increased interactions aid in achieving and maintaining greater hair volume. The combination of the fluid-encapsulated, flexible microspheres with the microparticles and the polymer also provides improvement in hair-to-hair interactions and consequently greater hair volume.

The present invention further relates to methods for enhancing hair volume, and more particularly for enhancing hair volume with leave-in aqueous cosmetic compositions which contain non-spherical microparticles of less than 100 μm in its longest dimension and a water-soluble or water-swellable polymer, and optionally fluid-encapsulated, flexible microspheres exhibiting a mean particle size of less than about 300 μm in diameter.

### DETAILED DESCRIPTION OF THE INVENTION

The leave-in hair cosmetic compositions of the present invention comprise select non-spherical microparticles in combination with a water-soluble or water-swellable polymer and may further comprise select fluid-encapsulated, flexible microspheres. Each of these essential components, as well as additional preferred or optional components, are described in detail hereinafter.

All percentages, parts and ratios are based upon the total weight of the compositions of the present invention, unless otherwise specified. All such weights as they pertain to listed ingredients are based on the active level and, therefore, do not include solvents or by-products that may be included in commercially available materials, unless otherwise specified.

All molecular weights as used herein are weight average molecular weights expressed as grams/mole, unless otherwise specified.

The term “spherical” as used herein, refers to a body which is the set of points in a metric space whose distance from a fixed point is approximately constant. Here, the meaning of “approximately” is that the fixed points are within a distance of ±15%. Therefore, “non-spherical”, as used herein, refers to a body whose surface dimensions vary in excess of ±15%.

The term “leave-in” as used herein, means that the product, after application unto hair, is not removed from the hair.

The term “fluid” as used herein, means a liquid or a gas which tends to take the shape of its container, container being the wall of the flexible microspheres.

The term “fluid-encapsulated” as used herein, means that the microspheres of the invention are structurally hollow. In accordance with the invention, the term “structurally hollow” nonetheless allows the hollow microspheres to contain at least one additional material therein.

The term “continuous or semi-continuous film network” as used herein, means that the film feels dry when touched with fingers, can be lifted easily from the substrate, and it does not melt when held between the fingers.

The term “suitable for application to human hair” as used herein, means that the compositions or components thereof so described are suitable for use in contact with human hair and the scalp and skin without undue toxicity, incompatibility, instability, allergic response, and the like.

The term “water soluble” as used herein, means that the polymer is soluble in water in the present composition. In general, the copolymer should be soluble at 25° C. at a concentration of 0.1% by weight of the water solvent, preferably at 1%, more preferably at 5%, most preferably at 15%.

The term “water swellable” as used herein, means that polymer can absorb sufficient amounts of water. In general a sufficient amount of water absorption means a water absorption of at least 1 ml/g, preferably at least 5 ml/g, more preferably at least 10 g/ml, most preferably at least 15 g/ml.

All cited references are incorporated herein by reference in their entireties. Citation of nay reference is not an admission regarding any determination as to its availability as prior art to the claimed invention.

### Microparticles

The microparticles of the present invention have a size of less than about 100 μm in its longest dimension. Preferably, the microparticles range from about 1 μm to about 60 μm, more preferably from about 1 μm to about 20 μm, and most preferably from about 1 μm to about 10 μm in its longest dimension.

The microparticles of the present invention may be natural, processed or synthetic clays. Non-limiting examples of commercially available suitable natural clay microparticles are silicas, borosilicates and silicons such as Smectite, Bentonite, Laponite and Megnabrite clays. Particularly preferred natural clay microparticles are Bentone Mass. Non-limiting examples of commercially available suitable synthetic microparticles are crosslinked polymeric ion exchange resins, cationic and anionic. The clays may be selected from smectite, attapulgite and sepiolite clays. Please refer to Mineralogical Society Monograph No.5 “Crystal Structures of Clay Minerals and their X-ray identification”, Brindley, G. et al., 1980, for an identification of these clays and their constituent groups. Processed clays are natural clays which have been processed to reduce impurities or to alter the balance of their constituents. Synthetic clays having equivalent structures may be used. Synthetic clays may have the advantage of being made from pure materials, thus avoiding the presence of impurities which can cause gassing or other problems.

The smectite mineral known as hectorite is a magnesium silicate in which the anionic silicate may or may not contain lithium and/or fluorine atoms and is charge balanced by sodium or other cations. The hectorites useful in the present invention include synthetic and natural minerals including magnesium silicate, sodium magnesium fluorosilicate, sodium lithium magnesium silicate and sodium lithium magnesium fluorosilicate. The preparation of synthetic hectorites by precipitation techniques is described in patent number GB 105411, GB 1213122 and U.S. Pat. No. 4,049,780, all incorporated herein. Synthetic hectorite is available commercially as Laponite™ from Southern Clay Products. The synthetic hectorite following the general empirical formula may be suitable for use in the present invention:

[Si₈(MgₐLi_{b}H_{c})O₂₀(OH)_{4-y}F_{y]}_{z}zM₊

wherein a=4.95 to 5.7, b=0 to 1.05, c=0 to 2, a+b+c=4 to 8, y=0 to 4, z=(12-2a-b-c), and M is Na⁺, Li⁺ or another charge balancing cation.

The surface of the microparticles of the present invention can be modified with a charge or at least one functional group that is hydrophobic or hydrophilic or a combination thereof. The surface charge can be through a static development or with the attachment of various ionic groups directly or linked via short, long or branched alkyl groups. The surface charge can be anionic, cationic, zwitterionic or amphoteric in nature. The functional groups comprise alkyl, hydroxy, alkoxy, amino, carboxy, sulfate and halide groups.

The microparticles of the present invention exist in either dry or hydrated state.

The aforesaid materials are nontoxic and non-irritating to the skin.

In the compositions of the present invention, it is preferable to incorporate from about 0.25% to about 15%, by weight of the composition, more specifically from about 0. 1% to about 10% by weight of the composition, of the microparticles, more preferably from about 0.5% to about 5% by weight, and even more preferably from about 0.5% to about 3%, by weight of the composition.

### Water-Soluble or Water-Swellable Polymer

The polymers useful in this invention may comprise any water-soluble or water-swellable polymer suitable for use in personal care products and for application to human hair. The polymers may be homopolymers, copolymers or a blend of homopolymers and/or copolymers. The polymers can be natural, synthetic, or semi-synthetic. Polymers can be straight chain or cross-linked. Polymers containing either ionic and non-ionic groups are contemplated. Ionic polymers include, but are not limited to, cationic, anionic, zwitterionic, and amphoteric polymers. The polymers can be synthesized from a variety of monomers containing unsaturated groups or by synthetic mechanisms that result in a variety of linking groups, for example, polyurethanes, polyesters, polyamides, polyureas, in the polymer backbone. The polymers of the present invention have a weight average molecular weight of at least about 5,000. There is no upper limit for molecular weight except that which limits applicability of the invention for practical reasons, such as viscosity, processing, aesthetic characteristics, formulation compatibility, etc. The weight average molecular weight is less than about 5,000,000, more generally less than about 2,500,000, and typically less than about 1,500,000. Preferably, the weight average molecular weight is from about 10,000 to about 5,000,000, more preferably from about 75,000 to about 1,000,000, even more preferably from about 100,000 to about 850,000, and most preferably from about 125,000 to about 750,000.

Examples of polymers for use in the present invention include straight polymer chains consisting of one or more monomers. A polymer containing two monomers can be represented by the following formula:

[A]ₐ[B]_{b}

wherein A and B are described herein; a is an integer of 1 or greater; and b is an integer of 0 or greater.

Monomer “A” Units

The “A” monomer unit is selected from polymerizable monomers, preferably ethylenically unsaturated monomers. By “polymerizable”, as used herein, it is meant that the monomers can be polymerized using any conventional synthetic techniques. Monomers that are polymerizable using conventional free radical initiated techniques are preferred. The term “ethylenically unsaturated” is used herein to mean monomers that contain at least one polymerizable carbon-carbon double bond (which can be mono-, di-, tri-, or tetra-substituted).

The ethylenically unsaturated A monomer units preferably can be described by the following formula

X−C(O)−CR⁵=CHR⁶

wherein X is selected from the group consisting of —OH, —OM, —OR⁴, —NH₂, —NHR⁴, and —N(R⁴)₂; M is a cation selected from the group consisting of Na+, K+, Mg++, Ca++, Zn++, NH₄+, alkylammonium, dialkylammonium, trialkylammonium, and tetralkylammonium; each R⁴ is independently selected from the group consisting of H, C₁-C₈ straight or branched chain alkyl, N,N-dimethylaminoethyl, methyl quaternized N,N-dimethyl-aminoethyl, 2-hydroxyethyl, 2-methoxyethyl, and 2-ethoxyethyl; and R⁵ and R⁶ are independently selected from the group consisting of H, C₁-C₈ straight or branched chain alkyl, methoxy, ethoxy, 2-hydroxyethoxy, 2-methoxyethyl, and 2-ethoxyethyl.

Representative nonlimiting examples of monomers useful herein include acrylic acid and salts, esters, and amides thereof. The salts can be derived from any of the common nontoxic metal, ammonium, or substituted ammonium counter ions. The esters can be derived from C₁-C₄₀ straight chain, C₃-C₄₀ branched chain, or C₃-C₄₀ carbocyclic alcohols; from polyhydric alcohols having from about 2 to about 8 carbon atoms and from about 2 to about 8 hydroxy groups (nonlimiting examples of which include ethylene glycol, propylene glycol, butylene glycol, hexylene glycol, glycerin, and 1,2,6-hexanetriol); from amino alcohols (nonlimiting examples of which include aminoethanol, dimethylaminoethanol, and diethylaminoethanol, and their quaternized derivatives); or from alcohol ethers (nonlimiting examples of which include methoxyethanol, and ethoxy ethanol). The amides can be unsubstituted, N-alkyl or N-alkylamino mono-substituted, or N,N-dialkyl or N,N-dialkylamino di-substituted, wherein the alkyl or alkylamino group can be derived from C₁-C₄₀ straight chain, C₃-C₄₀ branched chain, or C₃-C₄₀ carbocyclic moieties. Additionally, the alkylamino groups can be quaternized. Also useful as monomers are substituted acrylic acids and salts, esters, and amides thereof, wherein the substituents are on the two and three carbon positions of the acrylic acid and are independently selected from the group consisting of C₁₋₄ alkyl, —CN, —COOH (e.g., methacrylic acid, ethacrylic acid, and 3-cyano acrylic acid). The salts, esters, and amides of these substituted acrylic acids can be defined as described above for the acrylic acid salts, esters, and amides. Other useful monomers include vinyl and allyl esters of C₁₋₄₀ straight chain, C₃-₄₀ branched chain, or C₃₋₄₀ carbocyclic carboxylic acids; vinyl and allyl halides (e.g., vinyl chloride and allyl chloride); vinyl and allyl substituted heterocyclic compounds (e.g., vinyl pyridine and allyl pyridine); vinylidene chloride; and hydrocarbons having at least one carbon-carbon double bond (e.g., styrene, alpha-methylstyrene, t-butylstyrene, butadiene, isoprene, cyclohexadiene, ethylene, propylene, 1-butene, 2-butene, isobutylene, vinyl toluene); and mixtures thereof. Other useful monomers are maleic anhydride, itaconic acid, fumaric acid, and crotonic acid.

Monomer “B” Units

B monomer units can be selected from the group comprising A monomer units or macromonomer units or a combination of the two. A macromonomer is a large polymeric type of monomer unit which can be further polymerized with itself, with other conventional monomers, or with other macromonomers. The term “macromonomer” is one that is familiar to the polymer chemist of ordinary skill in the art. Representative examples of various types of macromonomer units are listed in U.S. Pat. Nos. 5,622,694, 5,632,998, 5,919,439 and 5,929,173.

Examples of useful commercially available synthetic polymers are listed below. The names described are according to the nomenclature developed by the Cosmetic, Toiletry, and Fragrance Association, Inc. (CTFA). In few cases, where CTFA name is not available, chemical name is written. Non-limiting examples are: vinylcaprolactam/PVP/dimethylamino-ethylmethacrylate copolymer (trade name: Gaffic™, H20LD, ISP Corp.), vinyl acetate/crotonic acid/vinyl propionate copolymer (trade name: Luviset™, BASF), vinyl acetate/crotonates copolymer (trade name: Resyn™, National Starch Corp.), vinyl acetate/butyl maleate/isobomyl acrylate copolymer (trade name: Advantage CPV™; ISP), tyrene/vinylpyrrolidone copolymer (trade name: Polectron™, ISP); vinylpyrrolidone/vinyl acetate copolymers (ISP, BASF); polyvinylpyrrolidone/polyurethane interpolymer (Pecogel™, Phoenix); octylacrylamide/acrylates/butylaminoethylmethacrylate copolymer (Amphomer™, National Starch; quaternized poly-(vinylpyrrolidone/dimethylaminoethyl methacrylate (Polyquaternium-11; ISP), vinylpyrrolidone/ vinyl acetate/vinyl propionate copolymer (Luviskol™, BASF). In addition, other commercially available polymers listed in the Encyclopedia of Polymers and Thickeners, Cosmetic and Toiletries, page 95, Vol. 108, May 1993 are included in this invention.

Examples of natural and modified natural polymers are: copolymer of hydroxyethyl-cellulose and dimethyldiallyl ammonium chloride (Polyquaternium-4; National Starch), hydroxyethyl-cellulose (Natrosol™; Aqualon), xanthan gum (Calgon), and other polymers listed in the Encyclopedia of Polymers and Thickeners, Cosmetic and Toiletries, page 95, Vol. 108, May 1993 are included in this invention.

Useful polymers include silicone graft copolymers as listed in U.S. Pat. Nos. 5,565,193 and 5,622,694; hydrophobic graft copolymers as listed in U.S. Pat. No. 5,622,694; silicone block copolymers as listed in U.S. Pat. No. 6,074,628.

The water-soluble or water-swellable polymers of the present invention may also encompass carboxylic acid/carboxylate copolymers. The carboxylic acid/carboxylate copolymers herein are hydrophobically-modified cross-linked copolymers of carboxylic acid and alkyl carboxylate, and have an amphophilic property. These carboxylic acid/carboxylate copolymers are obtained by copolymerizing (1) a carboxylic acid monomer such as acrylic acid, methacrylic acid, maleic acid, maleic anhydride, itaconic acid, fumaric acid, crotonic acid, or α-chloroacrylic acid, (2) a carboxylic ester having an alkyl chain of from 1 to about 30 carbons, and preferably (3) a crosslinking agent of the following formula:

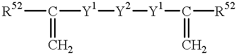

wherein R⁵² is a hydrogen or an alkyl group having from about 1 to about 30 carbons; y¹, independently, is oxygen, CH₂O, COO, OCO,

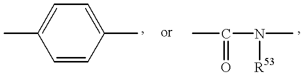

wherein R⁵³ is a hydrogen or an alkyl group having from about 1 to about 30 carbons; and y² is selected from (CH₂)ₘ″, (CH₂CH₂O)ₘ″, or (CH₂CH₂CH₂O)m” wherein m” is an integer of from 1 to about 30. The carboxylic acid/carboxylate copolymers herein are believed to provide appropriate viscosity and rheology properties to the composition, and to emulsify and stabilize certain conditioning agents in the composition. In the presence of microspheres, these polymers also aid in a solid film formation. It is further believed that, because of the alkyl group contained in the copolymer, the carboxylic acid/carboxylate copolymers do not make the composition undesirably sticky.

Suitable carboxylic acid/carboxylate copolymers herein are acrylic acid/alkyl acrylate copolymers having the following formula:

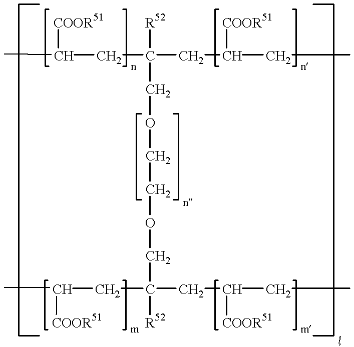

wherein R⁵¹, independently, is a hydrogen or an alkyl of 1 to 30 carbons wherein at least one of R⁵¹ is a hydrogen, R⁵² is as defined above, n, n′, m and m′ are integers in which n+n′+m+m′ is from about 40 to about 100, n″ is an integer of from 1 to about 30, and l is defined so that the copolymer has a molecular weight of about 500,000 to about 3,000,000.

Commercially available carboxylic acid/carboxylate copolymers useful herein include: CTFA name Acrylates/C₁₀-₃₀ Alkyl Acrylate Crosspolymer having tradenames Pemulene TR-1™, Pemulene TR-2™, Carbopol ₁₃₄₂™, Carbopol 1382™, and Carbopol ETD 2020™, all available from B. F. Goodrich Company.

Neutralizing agents may be included to neutralize the carboxylic acid/carboxylate copolymers herein. Nonlimiting examples of such neutralizing agents include sodium hydroxide, potassium hydroxide, ammonium hydroxide, monethanolamine, diethanolamine, triethanolamine, diisopropanolamine, aminomethylpropanol, tromethamine, tetrahydroxypropyl ethylenediamine, and mixtures thereof.

The concentration of the water-soluble or water-swellable polymer typically ranges from about 0.01% to about 10%, preferably from about 0.05% to about 5%, more preferably from about 0. 1% to about 2%, by weight of the composition.

### Aqueous Carrier

The compositions of the present invention comprise an aqueous carrier. The level and species of the carrier are selected according to the compatibility with other components, and other desired characteristic of the product.

Carriers useful in the present invention include water and water solutions of lower alkyl alcohols. Lower alkyl alcohols useful herein are monohydric alcohols having 1 to 6 carbons, more preferably ethanol and isopropanol.

Preferably, the aqueous carrier is substantially water. Deionized water is preferably used. Water from natural sources containing mineral cations can also be used, depending on the desired characteristic of the product. Generally, the compositions of the present invention comprise from about 20% to about 99%, preferably from about 40% to about 98%, and more preferably from about 60% to about 98% aqueous carrier.

The pH of the present composition is preferably from about 4 to about 9, more preferably from about 4.5 to about 7.5. Buffers and other pH adjusting agents can be included to achieve the desirable pH.

### Optional Components

Microspheres

In one embodiment, the leave-in hair cosmetic compositions of the present invention further comprise fluid-encapsulated, flexible microspheres. The microspheres are structurally hollow, however, they may contain various fluids, which encompass liquids and gases and their isomers. The gases include, but are not limited to, butane, pentane, air, nitrogen, oxygen, carbon dioxide, and dimethyl ether. If used, liquids may only be partially filled in the microspheres. The liquids include water and any compatible solvent. The liquids may also contain vitamins, amino acids, proteins and protein derivatives, herbal extracts, pigments, dyes, antimicrobial agents, chelating agents, UV absorbers, optical brighteners, silicone compounds, perfumes, humectants which are generally water soluble, additional conditioning agents which are generally water insoluble, and mixtures thereof. In one embodiment, water soluble components are preferred encompassed material. In another embodiment, components selected from the group consisting of vitamins, amino acids, proteins, protein derivatives, herbal extracts, and mixtures thereof are preferred encompassed materials. In yet another embodiment, components selected from the group consisting of vitamin E, pantothenyl ethyl ether, panthenol, Polygonum multiflori extracts, and mixtures thereof are preferred encompassed materials.

The microspheres typically have a size of less than about 300 μm in diameter. Preferably, the microspheres range from about 4 μm to about 200 μm, more preferably from about 5 μm to about 100 μm, and most preferably from about 8 μm to about 60 μm in diameter.

The microspheres typically have a density ranging from about 5 kg/m³ to about 200 kg/m³ and, preferably, greater than about 10 kg/m³ and/or less than about 100 kg/m³, and in particular ranging from about 15 kg/m³ to about 80 kg/m³. Microspheres of such low densities provide greater volume enhancement.

The fluid-encapsulated microspheres can have surface charges or their surface can be modified with organic or inorganic materials such as surfactants, polymers, and inorganic materials. Microsphere complexes are also useful. Non-limiting examples of complexes of gas-encapsulated microspheres are DSPCS-I2™ (silica modified ethylene/methacrylate copolymer microsphere) and SPCAT-I2™ (talc modified ethylene/methacrylate copolymer microsphere). Both of these are available from Kobo Products, Inc.

The surface of the microsphere may be charged through a static development or with the attachment of various ionic groups directly or linked via short, long or branched alkyl groups. The surface charge can be anionic, cationic, zwitterionic or amphoteric in nature.

The wall of the microspheres of the present invention is typically formed from a thermoplastic material. The thermoplastic material may be a polymer or copolymer of at least one monomer selected from the following groups: acrylates, methacrylates, styrene, substituted styrene, unsaturated dihalides, acrylonitriles, methacrylonitrile. The thermoplastic materials may contain amide, ester, urethane, urea, ether, carbonate, acetal, sulfide, phosphate, phosphonate ester, and siloxane linkages. In one embodiment, the microspheres comprise from 1% to 60% of recurring structural units derived from vinylidene chloride, from 20% to 90% of recurring structural units derived from acrylonitrile and from 1% to 50% of recurring structural units derived from a (meth)acrylic monomer, the sum of the percentages (by weight) being equal to 100. The (meth)acrylic monomer is, for example, a methyl acrylate or methacrylate, and especially the methacrylate. Preferably, the microspheres comprise of a polymer or copolymer of at least one monomer selected from expanded or non-expanded vinylidene chloride, acrylic, styrene, and (meth)acrylonitrile. More preferably, the microspheres comprise of a copolymer of acrylonitrile and methacrylonitrile.

Microspheres comprising polymers and copolymers obtained from esters, such as, for example, vinyl acetate or lactate, or acids, such as, for example, itaconic, citraconic, maleic or fumaric acids may also be used. See, in this regard, Japanese Patent Application No. JP-A-2-112304, the full disclosure of which is incorporated herein by reference.

Non-limiting examples of commercially available suitable low density microspheres are 551 DE (particle size range of approximately 30-50 μm and density of approximately 42 kg/m³), 551 DE 20 (particle size range of approximately 15-25 μm and density of approximately 60 kg/m³), 551 DE (particle size of approximately 40 μm and density of approximately 42 kg/m³ ), 461 DE (particle size range of approximately 20-40 μm and density 60 kg/m³), 551 DE 80 (particle size of approximately 50-80 μm and density of approximately 42 kg/m³ ), 091 DE (particle size range of approximately 35-55 μm and density of approximately 30 kg/m³), all of which are marketed under the trademark EXPANCEL™ by Akzo Nobel. PM 6545 (particle size range of approximately 110 μm and density of approximately 10 kg/M³ ), marketed under the trademark Plastic Microspheres™ by PQ Corporation, is another example of suitable microspheres for use herein. Particularly preferred microspheres are 551 DE 20, 551 DE 50 and 6545. The microspheres of the present invention exist in either dry or hydrated state. The aforesaid copolymers are nontoxic and non irritating to the skin.

The microspheres can be prepared, for example, via the processes described in EP-56,219, EP-348,372, EP-486,080, EP-320,473, EP-112,807 and U.S. Pat. No. 3,615,972, the full disclosure of each of which is incorporated herein by reference.

The wall of the microspheres is flexible. “Flexible”, as used herein, means that the microspheres are easy to compress. When pressure is reduced the microspheres regain their original volume. The flexible microspheres could alter their shape under an applied stress, or thermal expansion and contraction due to temperature change. Thus, the microspheres could expand upon heating. The volumizing benefits of the compositions of the present invention can be attributed to the flexibility of the microspheres.

The microspheres of the invention may be permeable or non-permeable. “Permeable”, as used herein, means that they permit a liquid to pass through them under given conditions.

In the embodiments of the present invention which include the micropheres, it is preferable to incorporate from about 0.25% to about 15%, by weight of the composition, more specifically from about 0.1% to about 10% by weight of the composition, of microspheres, more preferably from about 0.5% to about 5% by weight, and even more preferably from about 0.5% to about 2% of microspheres, by weight of the composition.

Amphoteric Conditioning Polymer

The compositions of the present invention may further contain an amphoteric conditioning polymer. The amphoteric conditioning polymers herein are those compatible with the carboxylic acid/carboxylate copolymers and which provide conditioning benefit to the hair. Although some of the amphoteric conditioning polymers herein may have some hair holding or hair fixative properties, such hair holding or hair fixative properties are not a requirement for the amphoteric conditioning polymers herein. The amphoteric conditioning polymers useful herein are those including at least one cationic monomer and at least one anionic monomer; the cationic monomer being quaternary ammonium, preferably dialkyl diallyl ammonium chloride or carboxylamidoalkyl trialkyl ammonium chloride; and the anionic monomer being carboxylic acid. The amphoteric conditioning polymers herein may include nonionic monomers such as acrylamine, methacrylate, or ethacrylate. Further, the amphoteric conditioning polymers useful herein do not contain betanized monomers.

The compositions of the present invention preferably comprise the amphoteric conditioning polymer at a level by weight of from about 0.01% to about 10%, more preferably from about 0.1% to about 5%.

Useful herein are polymers with the CTFA name Polyquaternium 22, Polyquaternium 39, and Polyquaternium 47. Such polymers are, for example, copolymers consisting of dimethyldiallyl ammonium chloride and acrylic acid, terpolymers consisting of dimethyldiallyl ammonium chloride and acrylamide, and terpolymers consisting of acrylic acid methacrylamidopropyl trimethyl-ammonium chloride and methyl acrylate such as those of the following formula wherein the ratio of n⁶:n⁷:n⁸ is 45:45:10.

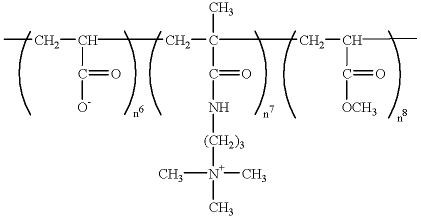

Highly preferred commercially available amphoteric conditioning polymers herein include Polyquaternium 22 with tradenames MERQUAT 280™, MERQUAT 295™, Polyquaternium 39 with tradenames MERQUAT PLUS 3330™, MERQUAT PLUS 3333™, and Polyquaternium 47 with tradenames MERQUAT 2001™, MERQUAT 2001N™, all available from Calgon Corporation.

Also useful herein are polymers resulting from the copolymerization of a vinyl monomer carrying at least one carboxyl group, such as acrylic acid, methacrylic acid, maleic acid, itaconic acid, fumaric acid, crotonic acid, or alphachloroacrylic acid, and a basic monomer which is a substituted vinyl compound containing at least one basic nitrogen atom, such as dialkylaminoalkyl methacrylates and acrylates and dialkylaminoalkylmethacrylamides and acrylarnides.

Also useful herein are polymers containing units derived from:

(i) at least one monomer chosen from amongst acrylamides or methacrylamides substituted on the nitrogen by an alkyl radical,

(ii) at least one acid comonomer containing one or more reactive carboxyl groups, and

(iii) at least one basic comonomer, such as esters, with primary, secondary and tertiary amine substituents and quaternary ammonium substituents, of acrylic and methacrylic acids, and the product resulting from the quaternization of dimethylaminoethyl methacrylate with dimethyl or diethyl sulfate.

The N-substituted acrylamides or methacrylamides which are most particularly preferred are the groups in which the alkyl radicals contain from 2 to 12 carbon atoms, especially N-ethylacrylamide, N-tert.-butylacrylamide, N-tert.-octylacrylamide, N-octylacrylamide, N-decyl-acrylamide and N-dodecylacrylamide and also the corresponding methacrylamides. The acid comonomers are chosen more particularly from amongst acrylic, methacrylic, crotonic, itaconic, maleic and fumaric acids and also the alkyl monoesters of maleic acid or fumaric acid in which alkyl has 1 to 4 carbon atoms.

The preferred basic comonomers are aminoethyl, butylaminoethyl, N,N′-dimethyl-aminoethyl and N-tert.-butylaminoethyl methacrylates.

Commercially available amphoteric conditioning polymers herein include octylacryl-amine/acrylates/butylaminoethyl methoacrylate copolymers with the tradenames AMPHOMER™, AMPHOMER SH701™, AMPHOMER 28-4910™, AMPHOMER LV71™, and AMPHOMER LV47™ supplied by National Starch & Chemical.

Thickening System

The compositions of the present invention may comprise a thickening system which comprises at least 2 thickening agents selected from the group consisting of a hydrophobically modified cellulose ether, an acrylate copolymer, and a crosslinked polymer, all described below. The thickening system useful herein is believed to provide improved conditioning benefits to the hair such as smoothness, softness, and reduction of friction, be easy to apply on the hair, and leave the hair and hands with a clean feeling.

The thickening system useful herein can also provide appropriate viscosity and rheology properties to the composition, so that the composition of the present composition has a suitable viscosity, preferably from about 1,000 cps to about 100,000 cps, more preferably from about 2,000 cps to about 50,000 cps. The viscosity herein can be suitably measured by Brookfield RVT at 20 rpm at 20° C. using either spindle #4, 5, 6 or 7 depending on the viscosity and the characteristic of the composition.

In view of providing improved conditioning benefits to the hair while leaving the hair and hands with a clean feeling, and also in view of providing appropriate viscosity and rheology properties, the thickening systems of the composition of the present invention preferably comprise all of these 3 thickening agents.

In view of providing improved conditioning benefits, in the composition of the present invention, the thickening system is preferably a nonionic or cationic system, more preferably a cationic system. The thickening system useful herein has improved compatibility with cationic hair conditioning agents. In the present invention, what is meant by a nonionic system is that the system comprises only nonionic thickening agents, but no cationic thickening agents. In the present invention, what is meant by a cationic system is that the system comprises at least one cationic thickening agent. The cationic system can include nonionic thickening agents. In such preferable nonionic or cationic thickening systems, the hydrophobically modified cellulose ether useful herein is preferably a nonionic thickening agent, and the acrylates copolymer and the crosslinked polymer useful herein are preferably independently a nonionic or cationic thickening agent. More preferably, the hydrophobically modified cellulose ether useful herein is a nonionic thickening agent, and the acrylates copolymer and the crosslinked polymer useful herein are cationic thickening agents. Cationic thickening agents useful herein may provide conditioning benefits.

In embodiments of the compositions which include a tickening sytem as described herein, the thickening system is typically included in the composition of the present invention at a level by weight of preferably from about 0.05% to about 10%, more preferably from about 0.1% to about 8%, still preferably from about 0.1% to about 5%.

Hydrophobically Modified Cellulose Ether

The composition of the present invention may comprise a hydrophobically modified cellulose ether as a thickening agent. The hydrophobically modified cellulose ether can be included in the composition of the present invention at a level by weight of preferably from about 0.01% to about 10%, more preferably from about 0.01% to about 5%, still more preferably from about 0.05% to about 2%.

The hydrophobically modified cellulose ethers useful herein are preferably nonionic polymers. The hydrophobically modified cellulose ethers useful herein comprise a hydrophilic cellulose backbone and a hydrophobic substitution group. The hydrophilic cellulose backbone has a sufficient degree of nonionic substitution to cellulose to be water soluble. Such hydrophilic cellulose backbone is selected from the group consisting of methyl cellulose, hydroxymethyl cellulose, hydroxyethyl cellulose, hydroxyethyl ethylcellulose, hydroxypropyl cellulose, hydroxypropyl methylcellulose, and mixtures thereof The amount of nonionic substitution is not critical, so long as there is an amount sufficient to assure that the hydrophilic cellulose backbone is water-soluble. The hydrophilic cellulose backbone has a molecular weight of about less than 800,000, preferably from about 20,000 to about 700,000, or from about 75 to about 2500. Further, where a high viscosity building effect is not desirable, a lower molecular weight cellulose backbone is preferred. One of the preferred hydrophilic cellulose backbone is hydroxyethyl cellulose having a molecular weight of from about 50,000 to about 700,000. Hydroxyethyl cellulose of this molecular weight is known to be one of the most hydrophilic of the materials contemplated. Thus, hydroxyethyl cellulose can be modified to a greater extent than other hydrophilic cellulose backbones.

The hydrophilic cellulose backbone is further substituted with a hydrophobic substitution group via an ether linkage to render the hydrophobically modified cellulose ether to have less than 1% water solubility, preferably less than 0.2% water solubility. The hydrophobic substitution group is selected from a straight or branched chain alkyl group of from about 10 to about 22 carbons; wherein the ratio of the hydrophilic groups in the hydrophilic cellulose backbone to the hydrophobic substitution group being from about 2:1 to about 1000:1, preferably from about 10:1 to about 100:1.

Commercially available hydrophobically modified cellulose ethers useful herein include: cetyl hydroxyethylcellulose having tradenames NATROSOL PLUS 330CS™ and POLYSURF 67™, both available from Aqualon Company, Del, USA, having cetyl group substitution of about 0.4% to about 0.65% by weight of the entire polymer.

Acrylate Copolymer

The compositions of the present invention may comprise an acrylate copolymer as a thickening agent. The acrylate copolymer can be included in the compositions of the present invention at a level by weight of preferably from about 0.01% to about 10%, more preferably from about 0.01% to about 5%, still more preferably from about 0.05% to about 2%.

The acrylate copolymers useful herein are preferably nonionic or cationic polymers, more preferably cationic polymers especially when the composition of the present invention has an acidic pH. The copolymers useful herein comprise by weight:

(a) from about 5% to about 80% of an acrylate monomer selected from the group consisting of a C₁-C₆ alkyl ester of acrylic acid, a C₁-C₆ alkyl ester of methacrylic acid, and mixtures thereof;

(b) from about 5% to about 80% of a monomer selected from the group consisting of a vinyl-substituted heterocyclic compound containing at least one of a nitrogen or sulfur atom, (meth)acrylamide, a mono- or di-(C₁-C₄)alkylamino(C₁-C₄)alkyl(meth)acrylate, a mono- or di-(C₁-C₄)alkylamino(C₁-C₄)alkyl(meth)acrylamide, and mixtures thereof; and

(c) from 0% to about 30% of an associative monomer.

The acrylate monomers (a) are selected from the group consisting of esters prepared from acrylic acid and C₁-C₆ alcohols such as methyl, ethyl, or propyl alcohol, and esters prepared from methacrylic acid and C₁-C₆ alcohols. Preferred are C₂-C₆ alkyl esters of acrylic acid, and more preferred is ethyl acrylate. The acrylate monomers (a) are included in the acrylates copolymer at a level by weight of from about 5% to about 80%, preferably from about 15% to about 70%, and more preferably from about 40% to about 70%.

The monomer (b) are selected from the group consisting of a vinyl-substituted heterocyclic compound containing at least one of a nitrogen or sulfur atom, (meth)acrylamide, a mono- or di-(C₁-C₄)alkylamino(C₁-C₄)alkyl(meth)acrylate, a mono- or di-(C₁-C₄)alkylamino(C₁-C₄)alkyl(meth)acryl-amide. Preferred are mono- or di-(C₁-C₄)alkylamino(C₁-C₄)alkyl(meth)-acrylates. Exemplary monomers (b) include N,N-dimethylamino ethyl methacrylate (DMAEMA), N,N-diethylamino ethyl acrylate, N,N-diethylamino ethyl methacrylate, N-t-butylamino ethyl acrylate, N-t-butylamino ethyl methacrylate, N,N-dimethylamino propyl acrylamide, N,N-dimethylamino propyl methacrylamide, N,N-diethylamino propyl acrylamide, and N,N-diethylamino propyl methacrylamide.

The monomers (b) are included in the acrylate copolymer at a level by weight of from about 5% to about 80%, preferably from about 10% to about 70%, and more preferably from about 20% to about 60%.

The associative monomers (c) are preferably selected from the group consisting of:

(i) urethane reaction products of a monoethylenically unsaturated isocyanate and nonionic surfactants comprising C₁-C₄ alkoxy-terminated, block copolymers of 1,2-butylene oxide and 1,2-ethylene oxide, as disclosed in U.S. Pat. No. 5,294,692;

(ii) an ethylenically unsaturated copolymerizable surfactant monomer obtained by condensing a nonionic surfactant with an acid, wherein the acid is selected from the group consisting of an α,β-ethylenically unsaturated carboxylic acid, anhydrides of α,β-ethylenically unsaturated carboxylic acids, and mixtures thereof, preferably, selected from the group consisting of a C₃-C₄ mono- or di-carboxylic acid, anhydrides of C₃-C₄ mono- or di-carboxylic acids, and mixtures thereof, more preferably, selected from the group consisting of acrylic acid, methacrylic acid, crotonic acid, maleic acid, maleic anhydride, itaconic acid, itaconic anhydride, and mixtures thereof, as disclosed in U.S. Pat. No. 4,616,074;

(iii) a surfactant monomer selected from the urea reaction product of a monoethylenically unsaturated monoisocyanate with a nonionic surfactant having amine functionality, as disclosed in U.S. Pat. No. 5,011,978;

(iv) an allyl ether of the formula: CH₂=CR′CH₂OAₘBₙAₚR, wherein R′ is hydrogen or methyl, A is propyleneoxy or butyleneoxy, B is ethyleneoxy, n is zero or an integer, m and p are independently zero or an integer less than n, and R is a hydrophobic group having at least 8 carbon atoms;

(v) a nonionic urethane monomer which is the urethane reaction product of a monohydric nonionic surfactant with a monoethylenically unsaturated isocyanate, preferably one lacking ester groups such as alpha, alpha-dimethyl-m-iso-propenyl benzyl isocyanate as disclosed in U.S. Pat. No. Re. 33,156; and

(vi) mixtures thereof.

Such associative monomers (c) include those disclosed in U.S. Pat. Nos. 3,657,175, 4,384,096, 4,616,074, 4,743,698, 4,792,343, 5,011,978, 5,102,936, 5,294,692, and Re. 33,156. Particularly preferred associative monomers (c) are those described in above (ii), i.e., the ethylenically unsaturated copolymerizable surfactant monomer obtained by condensing a nonionic surfactant with an acid, wherein the acid is selected from the group consisting of α,β-ethylenically unsaturated carboxylic acids, anhydrides of α,β-ethylenically unsaturated carboxylic acids, and mixtures thereof. More preferred associative monomers (c) are ethylenically unsaturated copolymerizable surfactant monomers obtained by condensing a nonionic surfactant with itaconic acid.

The associative monomers (c) are included in the acrylate copolymer at a level by weight of from 0% to about 30%, preferably from about 0.1% to about 10%.

In addition to required and preferred monomers discussed above, monomers which provide cross-linking in the polymer also may be utilized in relatively low amounts, preferably less than about 2%, more preferably from about 0.1% to about 1.0% by weight, based on the total weight of monomers used to prepare the polymer. Cross-linking monomers include multi-vinyl-substituted aromatic monomers, multi-vinyl-substituted alicyclic monomers, id-functional esters of phthalic acid, di-functional esters of methacrylic acid, multi-functional esters of acrylic acid, N-methylene-bis-acrylamide and multi-vinyl-substituted aliphatic monomers such as dienes, trienes, and tetraenes. Exemplary cross-linking monomers include divinylbenzene, trivinylbenzene, 1,2,4-trivinylcyclohexane, 1,5-hexadiene, 1,5,9-decatriene, 1,9-decadiene, 1,5-heptadiene, di-allyl phthalate, ethylene glycol dimethacrylate, polyethylene glycol dimethacrylate, penta- and tetra-acrylates, triallyl pentaerythritol, octaallyl sucrose, cycloparaffins, cycloolefins and N-methylene-bis-acrylamide. The polyethylene glycol dimethacrylates are preferred in view of thickening benefit particularly in aqueous compositions having an acidic pH.

Commercially available acrylate copolymers useful herein include: Acrylates/ Aminoacrylates/C₁₀₋₃₀Alkyl PEG-20 Itaconate copolymer having tradename Structure Plus available from National Starch.

Crosslinked Polymer

The compositions of the present invention may comprise a crosslinked polymer as a thickening agent. The crosslinked polymer can be included in the compositions of the present invention at a level by weight of preferably from about 0.01% to about 10%, more preferably from about 0.01% to about 5%, still more preferably from about 0.05% to about 2%.

Crosslinked polymers useful herein are generally described in U.S. Pat. Nos. 5,100,660, 4,849,484, 4,835,206, 4,628,078, 4,599,379, and EP 228,868, all of which are incorporated by reference herein in their entirety.

The crosslinked polymers useful herein are preferably nonionic or cationic polymers, more preferably cationic polymers. The crosslinked polymer useful herein comprises the monomer units and has the formula (A)ₘ(B)ₙ(C)ₚ wherein:

(A) is a dialkylaminoalkyl methacrylate, a quaternized dialkylaminoalkyl methacrylate, an acid addition salt of a quaternized dialkylaminoalkyl methacrylate, or mixtures thereof;

(B) is a dialkylaminoalkyl methacrylate, a quaternized dialkylaminoalkyl methacrylate, an acid addition salt of a quaternized dialkylaminoalkyl methacrylate, or mixtures thereof;

(C) is a nonionic monomer polymerizable with (A) or (B); and m, n, and p are independently zero or greater, but at least one of m or n is one or greater.

The monomer (C) can be selected from any of the commonly used monomers. Non-limiting examples of these monomers include ethylene, propylene, butylene, isobutylene, eicosene, maleic anhydride, acrylamide, methacrylamide, maleic acid, acrolein, cyclohexane, ethyl vinyl ether, and methyl vinyl ether. In the present invention, the monomer (C) is preferably acrylamide.

The alkyl portions of the monomers (A) and (B) are preferably short chain length alkyls such as C₁-C₈, more preferably C₁-C₅, still more preferably C₁-C₃, even still more preferably C₁-C₂. When quaternized, the polymers are preferably quaternized with short chain alkyls, i.e., C₁-C₈, more preferably C₁-C₅, still more preferably C₁-C₃, even still more preferably C₁-C₂. The acid addition salts refer to polymers having protonated amino groups. Acid addition salts can be performed through the use of halogen (e.g. chloride), acetic, phosphoric, nitric, citric, or other acids.

When the polymer contains the monomer (C), the molar proportion of the monomer (C) can be from 0% to about 99% based on the total molar proportions of the monomers (A), (B), and (C). The molar proportions of (A) and (B) can independently be from 0% to about 100%. When acrylamide is used as the monomer (C), it will preferably be included at a level of from about 20% to about 99%, more preferably from about 50% to about 99% based on the total molar proportions of the monomers (A), (B), and (C).

Where monomers (A) and (B) are both present, the molar ratio of monomer (A): monomer (B) in the final polymer is preferably from about 95:5 to about 15:85, more preferably from about 80:20 to about 20:80.

Where monomer (A) is not present and monomers (B) and (C) are both present, the molar ratio of monomer (B): monomer (C) in the final polymer is preferably from about 30:70 to about 70:30, more preferably from about 40:60 to about 60:40, still more preferably from about 45:55 to about 55:45.

The crosslinked polymers may also contain a crosslinking agent, which is typically a material containing two or more unsaturated functional groups. The crosslinking agent is reacted with the monomer units of the polymer and is incorporated into the polymer, forming either links or covalent bonds between two or more individual polymer chains or between two or more sections of the same polymer chain. Nonlimiting examples of suitable crosslinking agents include those selected from the group consisting of methylenebisacrylamides, diacrylates, dimethacrylates, di-vinyl aryl (e.g. di-vinyl phenyl ring) compounds, polyalkenyl polyethers of polyhydric alcohols, allyl acrylates, vinyloxyallkylacrylates, and polyfunctional vinylidenes. Specific examples of crosslinking agents useful herein include those selected from the group consisting of methylenebisacrylamide, ethylene glycol, propylene glycol, butylene glycol, di-(meth)acrylate, di-(meth)acrylamide, cyanomethylacrylate, vinyloxyethyleneacrylate, vinyloxyethylenemethacrylate, allyl pentaerythritol, trimethylolpropane, diallylether, allyl sucrose, butadiene, isoprene, 1,4-di-ethylene benzene, divinyl naphthalene, ethyl vinyl ether, methyl vinyl ether, and allyl acrylate. Other crosslinking agents include formaldehyde and glyoxal. Preferred herein is methylenebisacrylamide.

Widely varying amounts of the crosslinking agents can be employed depending upon the properties desired in the final polymer, e.g. viscosifying effect. The crosslinking agents will typically comprise from about 1 ppm to about 10,000 ppm, preferably from about 5 ppm to about 750 ppm, more preferably from about 25 ppm to about 500 ppm, even more preferably from about 100 ppm to about 500 ppm, and preferably from about 250 ppm to about 500 ppm of the total weight of the polymer on a weight/weight basis.

Examples of the crosslinked polymers useful herein include those conforming to the general structure (A)m(B)n(C)p wherein m is zero, (B) is methyl quaternized dimethylaminoethyl methacrylate, the molar ratio of monomers (B):(C) is about 45:55 to about 55:45, and the crosslinking agent is methylenebisacrylamide. An example of such a crosslinking polymer is one that is commercially available as a mineral oil dispersion (which can be include various dispersing aids such as PPG-1 trideceth-6) under the trademark Salcare® SC92 available from Allied Colloids Ltd. This polymer has the CTFA designation, “Polyquaternium 32 (and) Mineral Oil”.

Other crosslinked polymers useful herein include those not containing acrylamide or other monomer (C), i.e. p is zero. In these polymers, the monomers (A) and (B) are as described above. An especially preferred group of these polymers is one in which m is also zero. In this instance, the polymer is essentially a homopolymer of dialkylaminoalkyl methacrylate monomer or its quaternary ammonium or acid addition salt. These dialkylaminoalkyl methacrylate copolymers and homopolymers also contain a crosslinking agent as described above.

Preferably, the homopolymer which does not contain acrylamide or other monomer (C) is used in the composition of the present invention. The homopolymers useful herein can be those conforming to the general structure (A)ₘ(B)ₙ(C)ₚ wherein m is zero, (B) is methyl quaternized dimethylaminoethyl methacrylate, p is zero, and the crosslinking agent is methylenebis-acrylamide. An example of such a homopolymer is one that is commercially available as a mineral oil dispersion (which can include various dispersing aids such as PPG-1 trideceth-6) under the trademark Salcare® SC95 available from Allied Colloids Ltd. This polymer has the CTFA designation, “Polyquaternium 37 (and) Mineral Oil (and) PPG-1 Trideceth-6”. Another example of such a homopolymer is one that is commercially available as an ester dispersion, wherein the ester can be Propylene Glycol Dicaprylate/Dicaprate and the dispersion can include various dispersing aids such as PPG-1 trideceth-6, under the trademark Salcare® SC96 available from Allied Colloids Ltd. This polymer has the CTFA designation, “Polyquaternium 37 (and) Propylene Glycol Dicaprylate/Dicaprate (and) PPG-1 Trideceth-6”.

Silicone Compound

In one embodiment, the compositions of the present invention contain a silicone compound. The silicone compounds useful herein include volatile soluble or insoluble, or nonvolatile soluble or insoluble silicone conditioning agents. By “soluble” what is meant is that the silicone compound is miscible with the carrier of the composition so as to form part of the same phase. By insoluble what is meant is that the silicone forms a separate, discontinuous phase from the carrier, such as in the form of an emulsion or a suspension of droplets of the silicone. The silicone compounds herein may be made by any suitable method known in the art, including emulsion polymerization. The silicone compounds may further be incorporated in the present composition in the form of an emulsion, wherein the emulsion is made my mechanical mixing, or in the stage of synthesis through emulsion polymerization, with or without the aid of a surfactant selected from anionic surfactants, nonionic surfactants, cationic surfactants, and mixtures thereof.

The silicone compounds, when used herein, are preferably used at levels by weight of the composition of from about 0.1% to about 40%, more preferably from about 0.1% to about 10%, still more preferably from about 0.1% to about 5%.

A nonvolatile dispersed silicone that can be especially useful is a silicone gum. The term “silicone gum”, as used herein, means a polyorganosiloxane material having a viscosity at 25° C. of greater than or equal to 1,000,000 centistokes. It is recognized that the silicone gums described herein can also have some overlap with the above-disclosed silicone compounds. This overlap is not intended as a limitation on any of these materials. Silicone gums are described by Petrarch, and others including U.S. Pat. No. 4,152,416, to Spitzer et al., issued May 1, 1979 and Noll, Walter, Chemistry and Technology of Silicones, New York: Academic Press 1968. Also describing silicone gums are General Electric Silicone Rubber Product Data Sheets SE 30, SE 33, SE 54 and SE 76. The “silicone gums” will typically have a mass molecular weight in excess of about 200,000, generally between about 200,000 and about 1,000,000. Specific examples include polydimethylsiloxane, poly(dimethylsiloxane methylvinylsiloxane) copolymer, poly(dimethyl-siloxane diphenylsiloxane methylvinylsiloxane) copolymer and mixtures thereof.

Also useful are silicone resins, which are highly crosslinked polymeric siloxane systems. The crosslinking is introduced through the incorporation of tri-functional and tetra-functional silanes with mono-functional or di-functional, or both, silanes during manufacture of the silicone resin. As is well understood in the art, the degree of crosslinking that is required in order to result in a silicone resin will vary according to the specific silane units incorporated into the silicone resin. In general, silicone materials which have a sufficient level of trifunctional and tetrafunctional siloxane monomer units, and hence, a sufficient level of crosslinking, such that they dry down to a rigid, or hard, film are considered to be silicone resins. The ratio of oxygen atoms to silicon atoms is indicative of the level of crosslinking in a particular silicone material. Silicone materials which have at least about 1.1 oxygen atoms per silicon atom will generally be silicone resins herein. Preferably, the ratio of oxygen:silicon atoms is at least about 1.2:1.0. Silanes used in the manufacture of silicone resins include monomethyl-, dimethyl-, trimethyl-, monophenyl-, diphenyl-, methylphenyl-, monovinyl-, and methylvinylchlorosilanes, and tetrachlorosilane, with the methyl substituted silanes being most commonly utilized. Preferred resins are offered by General Electric as GE SS4230 and SS4267. Commercially available silicone resins will generally be supplied in a dissolved form in a low viscosity volatile or nonvolatile silicone fluid. The silicone resins for use herein should be supplied and incorporated into the present compositions in such dissolved form, as will be readily apparent to those skilled in the art. Without being bound by theory, it is believed that the silicone resins can enhance deposition of other silicone compounds on the hair and can enhance the glossiness of hair with high refractive index volumes.

Other useful silicone resins are silicone resin powders such as the material given the CTFA designation polymethylsilsequioxane, which is commercially available as Tospearl® from Toshiba Silicones.

The method of manufacturing these silicone compounds, can be found in Encyclopedia of Polymer Science and Engineering, Volume 15, Second Edition, pp. 204-308, John Wiley & Sons, Inc., 1989.

Silicone materials and silicone resins in particular, can conveniently be identified according to a shorthand nomenclature system well known to those skilled in the art as the “MDTQ” nomenclature. Under this system, the silicone is described according to the presence of various siloxane monomer units which make up the silicone. Briefly, the symbol M denotes the mono-functional unit (CH₃)₃SiO₀₅; D denotes the difunctional unit (CH₃)₂SiO; T denotes the trifunctional unit (CH₃)SiO₁₅; and Q denotes the quadri- or tetra-functional unit SiO2. Primes of the unit symbols, e.g., M′, D′, T′, and Q′ denote substituents other than methyl, and must be specifically defined for each occurrence. Typical alternate substituents include groups such as vinyl, phenyl, amino, hydroxyl, etc. The molar ratios of the various units, either in terms of subscripts to the symbols indicating the total number of each type of unit in the silicone, or an average thereof, or as specifically indicated ratios in combination with molecular weight, complete the description of the silicone material under the MDTQ system. Higher relative molar amounts of T, Q, T′ and/or Q′ to D, D′, M and/or or M′ in a silicone resin is indicative of higher levels of crosslinking. As discussed before, however, the overall level of crosslinking can also be indicated by the oxygen to silicon ratio.

The silicone resins for use herein which are preferred are MQ, MT, MTQ, MQ and MDTQ resins. Thus, the preferred silicone substituent is methyl. Especially preferred are MQ resins wherein the M:Q ratio is from about 0.5:1.0 to about 1.5:1.0 and the average molecular weight of the resin is from about 1000 to about 10,000.

The silicone compounds herein also include polyalkyl or polyaryl siloxanes with the following structure (I)

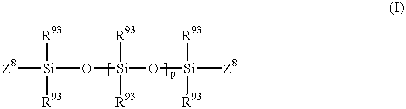

wherein R⁹³ is alkyl or aryl, and x is an integer from about 7 to about 8,000. Z⁸ represents groups which block the ends of the silicone chains. The alkyl or aryl groups substituted on the siloxane chain (R⁹³) or at the ends of the siloxane chains Z⁸ can have any structure as long as the resulting silicone remains fluid at room temperature, is dispersible, is neither irritating, toxic nor otherwise harmful when applied to the hair, is compatible with the other components of the composition, is chemically stable under normal use and storage conditions, and is capable of being deposited on and conditions the hair. Suitable Zs groups include hydroxy, methyl, methoxy, ethoxy, propoxy, and aryloxy. The two R⁹³ groups on the silicon atom may represent the same group or different groups. Preferably, the two R⁹³ groups represent the same group. Suitable R⁹³ groups include methyl, ethyl, propyl, phenyl, methylphenyl and phenylmethyl. The preferred silicone compounds are polydimethylsiloxane, polydiethylsiloxane, and polymethylphenylsiloxane. Polydimethyl-siloxane, which is also known as dimethicone, is especially preferred. The polyalkylsiloxanes that can be used include, for example, polydimethylsiloxanes. These silicone compounds are available, for example, from the General Electric Company in their Viscasil® and SF 96 series, and from Dow Corning in their Dow Coming 200 series.

Polyalkylaryl siloxane fluids can also be used and include, for example, polymethyl-phenylsiloxanes. These siloxanes are available, for example, from the General Electric Company as SF 1075 methyl phenyl fluid or from Dow Coming as 556 Cosmetic Grade Fluid.

Especially preferred for enhancing the shine characteristics of hair are highly arylated silicone compounds, such as highly phenylated polyethyl silicone having refractive index of about 1.46 or higher, especially about 1.52 or higher. When these high refractive index silicone compounds are used, they should be mixed with a spreading agent, such as a surfactant or a silicone resin, as described below to decrease the surface tension and enhance the film forming ability of the material.

The silicone compounds that can be used include, for example, a polypropylene oxide modified polydimethylsiloxane although ethylene oxide or mixtures of ethylene oxide and propylene oxide can also be used. The ethylene oxide and polypropylene oxide level should be sufficiently low so as not to interfere with the dispersibility characteristics of the silicone. These material are also known as dimethicone copolyols.

Other silicone compounds include amino substituted materials. Suitable alkylamino substituted silicone compounds include those represented by the following structure (II)

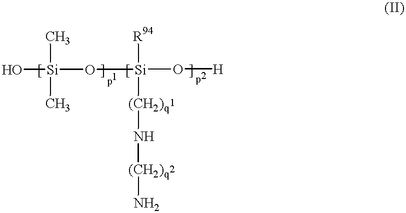

wherein R⁹⁴ is H, CH₃ or OH, p¹, p², q and q² are integers which depend on the molecular weight, the average molecular weight being approximately between 5,000 and 10,000. This polymer is also known as “amodimethicone”.

Suitable amino substituted silicone fluids include those represented by the formula (III)

(R⁹⁷)ₐG_{3—a}—Si—(OSiG₂)ₚ₃—(OSiG_{b}(R⁹⁷)_{2—b})ₚ₄—O—SiG_{3—a}(R⁹⁷)ₐ (3)

in which G is chosen from the group consisting of hydrogen, phenyl, OH, C₁-C₈ alkyl and preferably methyl; a denotes 0 or an integer from 1 to 3, and preferably equals 0; b denotes 0 or 1 and preferably equals 1; the sum p³+p⁴ is a number from 1 to 2,000 and preferably from 50 to 150, p³ being able to denote a number from 0 to 1,999 and preferably from 49 to 149 and p⁴ being able to denote an integer from 1 to 2,000 and preferably from 1 to 10; R⁹⁷ is a monovalent radical of formula Cq₃H_{2q3}L in which q³ is an integer from 2 to 8 and L is chosen from the groups

—N(R⁹⁶)CH₂—CH₂—N(R⁹⁶)₂

—N(R⁹⁶)₂

—N(R⁹⁶)₃X′

—N(R⁹⁶)CH₂—CH₂—NR⁹⁶H₂X′

in which R⁹⁶ is chosen from the group consisting of hydrogen, phenyl, benzyl, a saturated hydrocarbon radical, preferably an alkyl radical containing from 1 to 20 carbon atoms, and X′ denotes a halide ion.

An especially preferred amino substituted silicone corresponding to formula (II) is the polymer known as “trimethylsilylamodimethicone” wherein R⁹⁴ is CH₃.

Other amino substituted silicone polymers which can be used are represented by the formula (V):

[chemical structure]

where R⁹⁸ denotes a monovalent hydrocarbon radical having from 1 to 18 carbon atoms, preferably an alkyl or alkenyl radical such as methyl; R⁹⁹ denotes a hydrocarbon radical, preferably a C₁-C₁₈ alkylene radical or a C₁-C₁₈, and more preferably C₁-C₈, alkyleneoxy radical; Q⁻ is a halide ion, preferably chloride; p⁵ denotes an average statistical value from 2 to 20, preferably from 2 to 8; p⁶ denotes an average statistical value from 20 to 200, and preferably from 20 to 50. A preferred polymer of this class is available from Union Carbide under the name “UCAR SILICONE ALE 56.”

References disclosing suitable nonvolatile dispersed silicone compounds include U.S. Pat. Nos. 2,826,551, to Geen; 3,964,500, to Drakoff, issued Jun. 22, 1976; 4,364,837, to Pader; and British Pat. No. 849,433, to Woolston. “Silicon Compounds” distributed by Petrarch Systems, Inc., 1984, provides an extensive, though not exclusive, listing of suitable silicone compounds.

The silicone compounds for use herein will preferably have a viscosity of from about 1,000 to about 2,000,000 centistokes at 25° C., more preferably from about 10,000 to about 1,800,000, and even more preferably from about 100,000 to about 1,500,000. The viscosity can be measured by means of a glass capillary viscometer as set forth in Dow Corning Corporate Test Method CTM0004, Jul. 20, 1970. Silicone compound of high molecular weight may be made by emulsion polymerization. Suitable silicone fluids include polyalkyl siloxanes, polyaryl siloxanes, polyalkylaryl siloxanes, polyether siloxane copolymers, and mixtures thereof. Other nonvolatile silicone compounds having hair conditioning properties can also be used.

Particularly suitable silicone compounds herein are non-volatile silicone oils having a molecular weight of from about 200,000 to about 600,000 such as Dimethicone, and Dimethiconol. These silicone compounds can be incorporated in the composition as silicone oils solutions; the silicone oils being volatile or non-volatile.

Commercially available silicone compounds which are useful herein include Dimethicone with tradename DC345 available from Dow Corning Corporation, Dimethicone gum solutions with tradenames SE 30, SE 33, SE 54 and SE 76 available from General Electric, Dimethiconol with tradenames DCQ2-1403 and DCQ2-1401 available from Dow Coming Corporation, Mixture of Dimethicone and Dimethiconol with tradename DC1403 available from Dow Coming Corporation, and emulsion polymerized Dimethiconol available from Toshiba Silicone as described in GB application 2,303,857.

Humectant

The compositions of the present invention may contain a humectant. The humectants herein are selected from the group consisting of polyhydric alcohols, water soluble alkoxylated nonionic polymers, and mixtures thereof. The humectants, when used herein, are preferably used at levels by weight of the composition of from about 0.1% to about 20%, more preferably from about 0.5% to about 5%

Polyhydric alcohols useful herein include glycerin, sorbitol, propylene glycol, butylene glycol, hexylene glycol, ethoxylated glucose, 1, 2-hexane diol, hexanetriol, dipropylene glycol, erythritol, trehalose, diglycerin, xylitol, maltitol, maltose, glucose, fructose, sodium chondroitin sulfate, sodium hyaluronate, sodium adenosine phosphate, sodium lactate, pyrrolidone carbonate, glucosamine, cyclodextrin, and mixtures thereof.

Water soluble alkoxylated nonionic polymers useful herein include polyethylene glycols and polypropylene glycols having a molecular weight of up to about 1000 such as those with CTFA names PEG-200, PEG-400, PEG-600, PEG-1000, and mixtures thereof.

Commercially available humectants herein include: glycerin with tradenames STAR™ and SUPEROL™ available from The Procter & Gamble Company, CRODEROL GA7000™ available from Croda Universal Ltd., PRECERIN™ series available from Unichema, and a same tradename as the chemical name available from NOF; propylene glycol with tradename LEXOL PG-865/855™ available from Inolex, 1,2-PROPYLENE GLYCOL USP available from BASF; sorbitol with tradenames LIPONIC™ series available from Lipo, SORBO™, ALEX™, A-625™, and A-641™ available from ICI, and UNISWEET 70™, UNISWEET CONC™ available from UPI; dipropylene glycol with the same tradename available from BASF; diglycerin with tradename DIGLYCEROL™ available from Solvay GmbH; xylitol with the same tradename available from Kyowa and Eizai; maltitol with tradename MALBIT available from Hayashibara, sodium chondroitin sulfate with the same tradename available from Freeman and Bioiberica, and with tradename ATOMERGIC SODIUM CHONDROITIN SULFATE available from Atomergic Chemetals; sodium hyaluronate with tradenames ACTIMOIST available from Active Organics, AVIAN SODIUM HYALURONATE series available from Intergen, HYALURONIC ACID Na available from Ichimaru Pharcos; sodium adenosine phosphate with the same tradename available from Asahikasei, Kyowa, and Daiichi Seiyaku; sodium lactate with the same tradename available from Merck, Wako, and Showa Kako, cyclodextrin with tradenames CAVITRON available from American Maize, RHODOCAP series available from Rhone-Poulenc, and DEXPEARL available from Tomen; and polyethylene glycols with the tradename CARBOWAX series available from Union Carbide.

Additional Viscosity Modifier

The compositions of the present invention may contain an additional viscosity modifier. The additional viscosity modifiers herein are water soluble or water miscible polymers, have the ability to increase the viscosity of the composition, and are compatible with the carboxylic acid/carboxylate copolymers. The additional viscosity modifier is selected so that the composition of the present composition has a suitable viscosity, preferably from about 1,000 cps to about 100,000 cps, more preferably from about 2,000 cps to about 50,000cps. If such a viscosity is achieved without the additional viscosity modifier, the additional viscosity modifier may not be necessary. The viscosity herein can be suitably measured by Brookfield RVT at 20 rpm at 20° C. using either spindle #4, 5, 6 or 7 depending on the viscosity and the characteristic of the composition. The additional viscosity modifiers herein are preferably used at levels by weight of the composition of from about 0.001% to about 5%, more preferably from about 0.05% to about 3%.

Additional viscosity modifiers useful herein include anionic polymers and nonionic polymers. Useful herein are vinyl polymers such as cross linked acrylic acid polymers with the CTFA name Carbomer, cellulose derivatives and modified cellulose polymers such as methyl cellulose, ethyl cellulose, hydroxyethyl cellulose, hydroxypropyl methyl cellulose, nitro cellulose, sodium cellulose sulfate, sodium carboxymethyl cellulose, crystalline cellulose, cellulose powder, polyvinylpyrrolidone, polyvinyl alcohol, guar gum, hydroxypropyl guar gum, xanthan gum, arabia gum, tragacanth, galactan, carob gum, guar gum, karaya gum, carragheenin, pectin, agar, quince seed (Cydonia oblonga Mill), starch (rice, corn, potato, wheat), algae colloids (algae extract), microbiological polymers such as dextran, succinoglucan, pulleran, starch-based polymers such as carboxymethyl starch, methylhydroxypropyl starch, alginic acid-based polymers such as sodium alginate, alginic acid propylene glycol esters, acrylate polymers such as sodium polyacrylate, polyethylacrylate, polyacrylamide, polyethyleneimine, and inorganic water soluble material such as bentonite, aluminum magnesium silicate, laponite, hectonite, and anhydrous silicic acid.

Polyalkylene glycols having a molecular weight of more than about 1000 are useful herein. Useful are those having the following general formula:

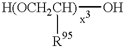

wherein R⁹⁵ is selected from the group consisting of H, methyl, and mixtures thereof. When R⁹⁵ is H, these materials are polymers of ethylene oxide, which are also known as polyethylene oxides, polyoxyethylenes, and polyethylene glycols. When R⁹⁵ is methyl, these materials are polymers of propylene oxide, which are also known as polypropylene oxides, polyoxypropylenes, and polypropylene glycols. When R⁹⁵ is methyl, it is also understood that various positional isomers of the resulting polymers can exist. In the above structure, ×3 has an average value of from about 1500 to about 25,000, preferably from about 2500 to about 20,000, and more preferably from about 3500 to about 15,000. Other useful polymers include the polypropylene glycols and mixed polyethylene-polypropylene glycols, or polyoxyethylene-polyoxypropylene copolymer polymers. Polyethylene glycol polymers useful herein are PEG-2M wherein R⁹⁵ equals H and ×3 has an average value of about 2,000 (PEG-2M is also known as Polyox WSR® N-10, which is available from Union Carbide and as PEG-2,000); PEG-5M wherein R⁹⁵ equals H and ×3 has an average value of about 5,000 (PEG-5M is also known as Polyox WSR® N-35 and Polyox WSR® N-80, both available from Union Carbide and as PEG-5,000 and Polyethylene Glycol 300,000); PEG-7M wherein R⁹⁵ equals H and ×3 has an average value of about 7,000 (PEG-7M is also known as Polyox WSR® N-750 available from Union Carbide); PEG-9M wherein R⁹⁵ equals H and ×3 has an average value of about 9,000 (PEG 9-M is also known as Polyox WSR® N-3333 available from Union Carbide); and PEG-14 M wherein R95 equals H and ×3 has an average value of about 14,000 (PEG-14M is also known as Polyox WSR® N-3000 available from Union Carbide).

Commercially available additional viscosity modifiers highly useful herein include Carbomers with tradenames Carbopol 934, Carbopol 940, Carbopol 950, Carbopol 980, and Carbopol 981, all available from B. F. Goodrich Company, acrylates/steareth-20 methacrylate copolymer with tradename ACRYSOL 22 available from Rohm and Hass, nonoxynyl hydroxyethylcellulose with tradename AMERCELL POLYMER HM-1500 available from Amerchol, methylcellulose with tradename BENECEL, hydroxyethyl cellulose with tradename NATROSOL, hydroxypropyl cellulose with tradename KLUCEL, cetyl hydroxyethyl cellulose with tradename POLYSURF 67, all supplied by Hercules, ethylene oxide and/or propylene oxide based polymers with tradenames CARBOWAX PEGs, POLYOX WASRs, and UCON FLUIDS, all supplied by Amerchol.

UV Absorber

The compositions of the present invention may further contain a UV (ultraviolet) absorber. Substantially transparent UV absorbers are particularly useful in the compositions herein. The UV absorbers are preferably used at levels of from about 0.01% to about 10%, by weight of the composition.

UV absorbers useful herein can be water soluble or water insoluble, including: p-aminobenzoic acid, its salts and its derivatives (ethyl, isobutyl, glyceryl esters; p-dimethylaminobenzoic acid); anhranilates (i.e., o-aminobenzoates; methyl, menthyl, phenyl, benzyl, phenylethyl, linalyl, terpinyl, and cyclohexenyl esters); salicylates (amyl, phenyl, benzyl, menthyl, glyceryl, and dipropyleneglycol esters); cinnamic acid derivatives (menthyl and benzyl esters, -phenyl cinnamonitrile; butyl cinnamoyl pyruvate; trihydroxycinnamic acid derivatives (esculetin, methylesculetin, daphnetin, and the glucosides, esculin and daphnin); dibenzalacetone and benzalacetophenone; naphtholsulfonates (sodium salts of 2-naphthol-3,6-disulfonic and of 2-naphthol-6,8-disulfonic acids); dihydroxy-naphthoic acid and its sals; o-and p-Hydroxybiphenyl-disulfonates; quinine salts (bisulfate, sulfate, chloride, oleate, and tannate); quinoline derivatives (8-hydroxyquinoline salts, 2-phenylquinoline); hydroxy- or methoxy-substituted benzophenones; uric and vilouric acids; tannic acid and its derivatives (e.g., hexaethylether); (butyl carbityl) (6-propyl piperonyl) ether; hydroquinone; benzophenones (oxybenzene, sulisobenzone, dioxybenzone, benzoresorcinol, 2,2′,4,4′-Tetrahydroxybenzo-phenone, 2,2′-Dihydroxy-4,4′-dimethoxybenzophenone, octabenzone); 4-Isopropyldibenzoyl-methane; butyl-methoxydibenzoyl-methane; etocrylene; and 4-isopropyl-di-benzoyl-methane. Of these, 2-ethylhexyl p-methoxy-cinnamate, 4,4′-t-butyl methoxy-dibenzoylmethane, 2-hydroxy-4-methoxybenzophenone, octyldimethyl p-aminobenzoic acid, digalloyltrioleate, 2,2-dihydroxy-4-methoxybenzophenone ethyl 4-[bis(hydroxypropyl)]-aminobenzoate, 2-ethylhexyl2-cyano-3,3-diphenylacrylate, 2-ethylhexyl-salicylate, glyceryl p-aminobenzoate, 3,3,5-trimethylcyclo-hexylsalicylate, methylanthranilate, p-dimethyl-amino-benzoic acid or amino-benzoate, 2-ethyl-hexyl p-dimethylamino-benzoate, 2-phenyl-benzimidazole-5-sulfonic acid, 2-(p-dimethylamino-phenyl)-5-sulfonicbenzoxazoic acid and mixtures thereof. Preferred sunscreens useful in the compositions of the present invention are 2-ethylhexyl p-methoxy-cinnamate, butyl-methoxydibenzoylmethane, 2-hydroxy-4-methoxy-benzo-phenone, octyl-dimethyl p-aminobenzoic acid and mixtures thereof.

Herbal Extracts

The compositions of the present invention may further contain herbal extracts. Herbal extracts useful herein include those which are water soluble and those which are water insoluble. Useful herbal extracts herein include: Polygonum multiflori Extract, Houttuynia cordate extract, Phellodendron Bark extract, melilot extract, white dead nettle extract, licorice root extract, herbaceous peony extract, soapwort extract, dishcloth gourd extract, cinchona extract, creeping saxifrage extract, Sophora angustifolia extract, candock extract, common fermel extract, primrose extract, rose extract, Rehmannia glutinosa extract, lemon extract, shikon extract, aloe extract, iris bulb extract, eucalyptus extract, field horsetail extract, sage extract, thyme extract, tea extract, laver extract, cucumber extract, clove extract, raspberry extract, melissa extract, ginseng extract, carrot extract, horse chestnut extract, peach extract, peach leaf extract, mulberry extract, cornflower extract, hamamelis extract, placenta extract, thymus extract, silk extract, algae extract, althea extract, angelica dahurica extract, apple extract, apricot kernel extract, arnica extract, Artemisia capillaris extract, astragal extract, balm mint extract, perilla extract, birch bark extract, bitter orange peel extract, Thea sinensis extract, burdock root extract, burnet extract, butcherbroom extract, Stephania cepharantha extract, matricaria extract, chrysanthemum flower extract, citrus unshiu peel extract, cnidium extract, coix seed extract, coltsfoot extract, comfrey leaf extract, crataegus extract, evening primrose oil, gambir extract, ganodermna extract, gardenia extract, gentian extract, geranium extract, ginkgo extract, grape leaf extract, crataegus extract, henna extract, honeysuckle extract, honeysuckle flower extract, hoelen extract, hops extract, horsetail extract, hydrangea extract, hypericum extract, isodonis extract, ivy extract, Japanese angelica extract, Japanese coptis extract, juniper extract, jujube extract, lady's mantle extract, lavender extract, lettuce extract, licorice extract, linden extract, lithospermum extract, loquat extract, luffa extract, malloti extract, mallow extract, calendula extract, moutan bark extract, mistletoe extract, mukurossi extract, mugwort extract, mulberry root extract, nettle extract, nutmeg extract, orange extract, parsley extract, hydrolyzed conchiorin protein, peony root extract, peppermint extract, philodendron extract, pine cone extract, platycodon extract, polygonatum extract, rehmannia extract, rice bran extract, rhubarb extract, rose fruit extract, rosemary extract, royal jelly extract, safflower extract, saffron crocus extract, sambucus extract, saponaria extract, Sasa albo marginata extract, Saxifraga stolonifera extract, scutellaria root extract, Cortinellus shiitake extract, lithospermum extract, sophora extract, laurel extract, calamus root extract, swertia extract, thyme extract, linden extract, tomato extract, turmeric extract, uncaria extract, watercress extract, logwood extract, grape extract, white lily extract, rose hips extract, wild thyme extract, witch hazel extract, yarrow extract, yeast extract, yucca extract, zanthoxylum extract, and mixtures thereof.

Commercially available herbal extracts useful herein include Polygonum multiflori extracts which are water soluble, and available from Institute of Occupational Medicine, CAPM, China National Light Industry, and Maruzen, and other herbal extracts listed above available from Maruzen.

Additional Conditioning Agent

The compositions of the present invention may further contain an additional conditioning agent selected from the group consisting of high melting point compounds, cationic surfactants, high molecular weight ester oils, cationic polymers, additional oily compounds, and mixtures thereof. Additional conditioning agents are selected according to the compatibility with other components, and the desired characteristic of the product. For example, components of cationic nature will be included in an amount which would not cause separation in view of the essential components of anionic nature. The additional conditioning agents herein are preferably used at levels by weight of the composition of from about 0.01% to about 10%.

High melting point compound

The high melting point compound useful herein have a melting point of at least about 25° C. selected from the group consisting of fatty alcohols, fatty acids, fatty alcohol derivatives, fatty acid derivatives, hydrocarbons, steroids, and mixtures thereof. It is understood by the artisan that the compounds disclosed in this section of the specification can in some instances fall into more than one classification, e.g., some fatty alcohol derivatives can also be classified as fatty acid derivatives. However, a given classification is not intended to be a limitation on that particular compound, but is done so for convenience of classification and nomenclature. Further, it is understood by the artisan that, depending on the number and position of double bonds, and length and position of the branches, certain compounds having certain required carbon atoms may have a melting point of less than about 25° C. Such compounds of low melting point are not intended to be included in this section. Nonlimiting examples of the high melting point compounds are found in International Cosmetic Ingredient Dictionary and Handbook, Eight Edition, 2000.

It is believed that these high melting point compounds cover the hair surface and reduce friction, thereby resulting in providing smooth feel on the hair and ease of combing.

The high melting point compound is preferably included in the composition at a level by weight of from about 0.01% to about 5%, more preferably from about 0.1% to about 1%. The weight of the carboxylic acid/carboxylate copolymer is preferably greater than about 0.5 times, more preferably 1.0 times, the weight of the high melting point compound.

The fatty alcohols useful herein are those having from about 14 to about 30 carbon atoms, preferably from about 16 to about 22 carbon atoms. These fatty alcohols can be straight or branched chain alcohols and can be saturated or unsaturated. Nonlimiting examples of fatty alcohols include, cetyl alcohol, stearyl alcohol, behenyl alcohol, and mixtures thereof.

The fatty acids useful herein are those having from about 10 to about 30 carbon atoms, preferably from about 12 to about 22 carbon atoms, and more preferably from about 16 to about 22 carbon atoms. These fatty acids can be straight or branched chain acids and can be saturated or unsaturated. Also included are diacids, triacids, and other multiple acids which meet the requirements herein. Also included herein are salts of these fatty acids. Nonlimiting examples of fatty acids include lauric acid, palmitic acid, stearic acid, behenic acid, sebacic acid, and mixtures thereof.

The fatty alcohol derivatives and fatty acid derivatives useful herein include alkyl ethers of fatty alcohols, alkoxylated fatty alcohols, alkyl ethers of alkoxylated fatty alcohols, esters of fatty alcohols, fatty acid esters of compounds having esterifiable hydroxy groups, hydroxy-substituted fatty acids, and mixtures thereof. Nonlimiting examples of fatty alcohol derivatives and fatty acid derivatives include materials such as methyl stearyl ether; the ceteth series of compounds such as ceteth-1 through ceteth-45, which are ethylene glycol ethers of cetyl alcohol, wherein the numeric designation indicates the number of ethylene glycol moieties present; the steareth series of compounds such as steareth-1 through 10, which are ethylene glycol ethers of steareth alcohol, wherein the numeric designation indicates the number of ethylene glycol moieties present; ceteareth 1 through ceteareth-10, which are the ethylene glycol ethers of ceteareth alcohol, i.e. a mixture of fatty alcohols containing predominantly cetyl and stearyl alcohol, wherein the numeric designation indicates the number of ethylene glycol moieties present; C₁-C₃₀ alkyl ethers of the ceteth, steareth, and ceteareth compounds just described; polyoxyethylene ethers of behenyl alcohol; ethyl stearate, cetyl stearate, cetyl palmitate, stearyl stearate, myristyl myristate, polyoxyethylene cetyl ether stearate, polyoxyethylene stearyl ether stearate, polyoxyethylene lauryl ether stearate, ethyleneglycol monostearate, polyoxyethylene monostearate, polyoxyethylene distearate, propyleneglycol monostearate, propyleneglycol distearate, trimethylolpropane distearate, sorbitan stearate, polyglyceryl stearate, glyceryl monostearate, glyceryl distearate, glyceryl tristearate, and mixtures thereof.

Hydrocarbons useful herein include compounds having at least about 20 carbons.

Steroids useful herein include compounds such as cholesterol.

High melting point compounds of a single compound of high purity are preferred. Single compounds of pure fatty alcohols selected from the group of pure cetyl alcohol, stearyl alcohol, and behenyl alcohol are highly preferred. By “pure” herein, what is meant is that the compound has a purity of at least about 90%, preferably at least about 95%. These single compounds of high purity provide good rinsibility from the hair when the consumer rinses off the composition.

Commercially available high melting point compounds useful herein include: cetyl alcohol, stearyl alcohol, and behenyl alcohol having tradenames KONOL™ series available from Shin Nihon Rika (Osaka, Japan), and NAA™ series available from NOF (Tokyo, Japan); pure behenyl alcohol having tradename I-DOCOSANOL™ available from WAKO (Osaka, Japan), various fatty acids having tradenames NEO-FAT™ available from Akzo (Chicago Ill., USA), HYSTRENE™ available from Witco Corp. (Dublin Ohio, USA), and DERMA™ available from Vevy (Genova, Italy); and cholesterol having tradename NIKKOL AGUASOME LA™ available from Nikko.

Cationic Surfactant

Among the cationic surfactants useful herein are those corresponding to the general formula (I):

[chemical structure]

wherein at least one of R⁷¹, R⁷², R⁷³ and R⁷⁴ is selected from an aliphatic group of from 8 to 30 carbon atoms or an aromatic, alkoxy, polyoxyalkylene, alkylamido, hydroxyalkyl, aryl or alkylaryl group having up to about 22 carbon atoms, the remainder of R⁷¹, R⁷², R⁷³ and R⁷⁴ are independently selected from an aliphatic group of from 1 to about 22 carbon atoms or an aromatic, alkoxy, polyoxyalkylene, alkylamido, hydroxyalkyl, aryl or alkylaryl group having up to about 22 carbon atoms; and X is a salt-forming anion such as those selected from halogen, (e.g. chloride, bromide), acetate, citrate, lactate, glycolate, phosphate, nitrate, sulfonate, sulfate, alkylsulfate, and alkyl sulfonate radicals. The aliphatic groups can contain, in addition to carbon and hydrogen atoms, ether linkages, and other groups such as amino groups. The longer chain aliphatic groups, e.g., those of about 12 carbons, or higher, can be saturated or unsaturated. Preferred is when R⁷¹, R⁷², R⁷³ and R⁷⁴ are independently selected from C₁ to about C₂₂ alkyl. Nonlimiting examples of cationic surfactants useful in the present invention include the materials having the following CTFA designations: quaternium-8, quaternium-14, quaternium-18, quaternium-18 methosulfate, quaternium-24, and mixtures thereof.

Among the cationic surfactants of general formula (I), preferred are those containing in the molecule at least one alkyl chain having at least 16 carbons. Nonlimiting examples of such preferred cationic surfactants include: behenyl trimethyl ammonium chloride available, for example, with tradename INCROQUAT TMC-80™ from Croda and ECONOL TM22™ from Sanyo Kasei; cetyl trimethyl ammonium chloride available, for example, with tradename CA-2350TM from Nikko Chemicals, hydrogenated tallow alkyl trimethyl ammonium chloride, dialkyl (14-18) dimethyl ammonium chloride, ditallow alkyl dimethyl ammonium chloride, dihydrogenated tallow alkyl dimethyl ammonium chloride, distearyl dimethyl ammonium chloride, dicetyl dimethyl ammonium chloride, di(behenyl/arachidyl) dimethyl ammonium chloride, dibehenyl dimethyl ammonium chloride, stearyl dimethyl benzyl ammonium chloride, stearyl propyleneglycol phosphate dimethyl ammonium chloride, stearoyl amidopropyl dimethyl benzyl ammonium chloride, stearoyl amidopropyl dimethyl (myristylacetate) ammonium chloride, and N-(stearoyl colamino formyl methy) pyridinium chloride.

Also preferred are hydrophilically substituted cationic surfactants in which at least one of the substituents contain one or more aromatic, ether, ester, amido, or amino moieties present as substituents or as linkages in the radical chain, wherein at least one of the R⁷¹-R⁷⁴ radicals contain one or more hydrophilic moieties selected from alkoxy (preferably C₁-C₃ alkoxy), polyoxyalkylene (preferably C₁-C₃ polyoxyalkylene), alkylamido, hydroxyalkyl, alkylester, and combinations thereof. Preferably, the hydrophilically substituted cationic conditioning surfactant contains from 2 to about 10 nonionic hydrophile moieties located within the above stated ranges. Preferred hydrophilically substituted cationic surfactants include those of the formula (II) through (VIII) below:

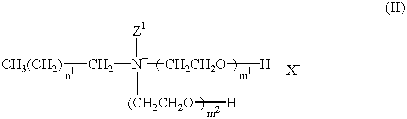

wherein n¹ is from 8 to about 28, m¹+m² is from 2 to about 40, Z¹ is a short chain alkyl, preferably a C₁-C₃ alkyl, more preferably methyl, or (CH₂CH₂O)ₘ₃H wherein m¹+m²+m³ is up to 60, and X is a salt forming anion as defined above;

[chemical structure]

wherein n² is 1 to 5, one or more of R⁷⁵, R⁷⁶, and R⁷⁷ are independently an C₁-C₃₀ alkyl, the remainder are CH₂CH₂OH, one or two of R⁷⁸, R⁷⁹, and R⁸⁰ are independently an C₁-C₃₀ alkyl, and remainder are CH₂CH₂OH, and X is a salt forming anion as mentioned above;

[chemical structure]

wherein, independently for formulae (IV) and (V), Z² is an alkyl, preferably C₁-C₃ alkyl, more preferably methyl, and Z³ is a short chain hydroxyalkyl, preferably hydroxymethyl or hydroxyethyl, n³ and n⁴ independently are integers from 2 to 4, inclusive, preferably from 2 to 3, inclusive, more preferably 2, R⁸¹ and R⁸², independently, are substituted or unsubstituted hydrocarbyls, C₁₂-C₂₀ alkyl or alkenyl, and X is a salt forming anion as defined above;

[chemical structure]

wherein R⁸³ is a hydrocarbyl, preferably a C₁-C₃ alkyl, more preferably methyl, Z⁴ and Z⁵ are, independently, short chain hydrocarbyls, preferably C₂-C₄ alkyl or alkenyl, more preferably ethyl, m⁴ is from 2 to about 40, preferably from about 7 to about 30, and X is a salt forming anion as defined above;

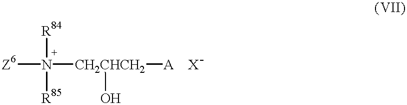

wherein R⁸⁴ and R⁵, independently, are C₁-C₃ alkyl, preferably methyl, Z⁶ is a C₁₂-C₂₂ hydrocarbyl, alkyl carboxy or alkylamido, and A is a protein, preferably a collagen, keratin, milk protein, silk, soy protein, wheat protein, or hydrolyzed forms thereof, and X is a salt forming anion as defined above;

[chemical structure]

wherein n⁵ is 2 or 3, R⁸⁶ and R⁸⁷, independently are C₁-C₃ hydrocarbyls preferably methyl, and X is a salt forming anion as defined above. Nonlimiting examples of hydrophilically substituted cationic surfactants useful in the present invention include the materials having the following CTFA designations: quaternium-16, quaternium-26, quaternium-27, quaternium-30, quaternium-33, quaternium-43, quaternium-52, quaternium-53, quaternium-56, quaternium-60, quaternium-61, quaternium-62, quaternium-70, quaternium-71, quaternium-72, quaternium-75, quaternium-76 hydrolyzed collagen, quaternium-77, quaternium-78, quaternium-79 hydrolyzed collagen, quaternium-79 hydrolyzed keratin, quaternium-79 hydrolyzed milk protein, quaternium-79 hydrolyzed silk, quaternium-79 hydrolyzed soy protein, and quaternium-79 hydrolyzed wheat protein, quaternium-80, quaternium-81, quaternium-82, quaternium-83, quaternium-84, and mixtures thereof.

Highly preferred hydrophilically substituted cationic surfactants include dialkylamido ethyl hydroxyethylmonium salt, dialkylamidoethyl dimonium salt, dialkyloyl ethyl hydroxyethylmonium salt, dialkyloyl ethyldimonium salt, and mixtures thereof; for example, commercially available under the following tradenames; VARISOFT 110™, VARISOFT 222™, VARIQUAT K1215™ and VARIQUAT 638™ from Witco Chemical, MACKPRO KLP™, MACKPRO WLW™, MACKPRO MLP™, MACKPRO NSP™, MACKPRO NLW™, MACKPRO WWPM, MACKPRO NLP™, MACKPRO SLP™ from McIntyre, ETHOQUAD 18/25™, ETHOQUAD 0/12PG™, ETHOQUAD C/25™, ETHOQUAD S/25™, and ETHODUOQUAD™ from Akzo, DEHYQUAT SP™ from Henkel, and ATLAS G265™ from ICI Americas.

Amines are suitable as cationic surfactants. Primary, secondary, and tertiary fatty amines are useful. Particularly useful are tertiary amido amines having an alkyl group of from about 12 to about 22 carbons. Exemplary tertiary amido amines include: stearamidopropyldimethylamine, stearamidopropyldiethylamine, stearamidoethyldiethylamine, stearamidoethyldimethylamine, palmitamidopropyldimethylamine, palmit-amidopropyldiethylamine, palmitamidoethyldiethylaminne, palmitamidoethyldimethyl-amine, behenamidopropyldimethylamine, behenamidopropyldiethylamine, behenamido-ethyldiethylamine, behenamidoethyldimethylamine, arachidamidopropyldimethylamine, arachidamidopropyldiethylamine, arachidamidoethyldiethylamine, arachidamidoethyl-dimethylamine, diethylaminoethylstearamide. Also useful are dimethylstearamine, dimethyl-soyamine, soyamine, myristylamine, tridecylamine, ethylstearylamine, N-tallowpropane diamine, ethoxylated (with 5 moles of ethylene oxide) stearylamine, dihydroxyethylstearylamine, and arachidylbehenylamine. Useful amines in the present invention are disclosed in U.S. Pat. No. 4,275,055, Nachtigal, et al.

These amines can also be used in combination with acids such as l-glutamic acid, lactic acid, hydrochloric acid, malic acid, succinic acid, acetic acid, fiumaric acid, tartaric acid, citric acid, l-glutamic hydrochloride, maleic acid, and mixtures thereof; more preferably l-glutamic acid, lactic acid, citric acid. The amines herein are preferably partially neutralized with any of the acids at a molar ratio of the amine to the acid of from about 1:0.3 to about 1:2, more preferably from about 1:0.4 to about 1:1.

High Molecular Weight Ester Oils

High molecular weight ester oils are useful herein. The high molecular weight ester oils useful herein are those which are water insoluble, have a molecular weight of at least about 500, preferably at least about 800, and are in liquid form at 25° C. Useful high molecular weight ester oils herein include pentaethytritol ester oils, trimethylol ester oils, poly α-olefin oils, citrate ester oils, glyceryl ester oils, and mixtures thereof. As used herein, the term “water insoluble” means the compound is substantially not soluble in water at 25° C.; when the compound is mixed with water at a concentration by weight of above 1.0%, preferably at above 0.5%, the compound is temporarily dispersed to form an unstable colloid in water, then is quickly separated from water into two phases.

The high molecular weight ester oil herein provides conditioning benefits such as moisturized feel, smooth feel, and manageability control to the hair when the hair is dried, yet not leave the hair feeling greasy. It is believed that water insoluble oily material in general are capable of being deposited on the hair. Without being bound by theory, it is believed that, because of its bulkiness, the high molecular weight ester oil covers the surface of the hair and, as a result, the high molecular weight ester oil reduces hair friction to deliver smoothness and manageability control to the hair. It is also believed that, because it has some hydrophilic groups, the high molecular weight ester oil provides moisturized feel, yet, because it is liquid, does not leave the hair feeling greasy. The high molecular weight ester oil is chemically stable under normal use and storage conditions.

Pentaerythritol ester oils useful herein are those having the following formula:

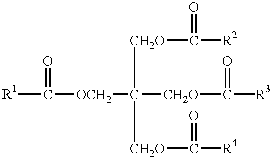

wherein R¹, R², R³, and R⁴, independently, are branched, straight, saturated, or unsaturated alkyl, aryl, and alkylaryl groups having from 1 to about 30 carbons. Preferably, R¹, R², R³, and R⁴, independently, are branched, straight, saturated, or unsaturated alkyl groups having from about 8 to about 22 carbons. More preferably, R¹, R², R³ and R⁴ are defined so that the molecular weight of the compound is from about 800 to about 1200.

Trimethylol ester oils useful herein are those having the following formula:

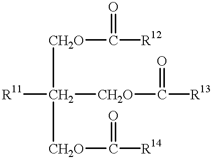

wherein R¹¹ is an alkyl group having from 1 to about 30 carbons, and R¹², R¹³, and R¹⁴, independently, are branched, straight, saturated, or unsaturated alkyl, aryl, and alkylaryl groups having from 1 to about 30 carbons. Preferably, R¹¹ is ethyl and R¹², R¹³, and R¹⁴, independently, are branched, straight, saturated, or unsaturated alkyl groups having from 8 to about 22 carbons. More preferably, R¹¹, R¹², R¹³ and R¹⁴ are defined so that the molecular weight of the compound is from about 800 to about 1200.

Poly α-olefin oils useful herein are those having the following formula and having a viscosity of from about 1 to about 35,000 cst, a molecular weight of from about 200 to about 60,000, and a polydispersity of no more than about 3;

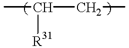

wherein R³¹ is an alkyl having from about 4 to 14 carbons, preferably 4 to 10 carbons. Poly α-olefin oils having a molecular weight of at least about 800 are useful herein. Such high molecular weight poly u.-olefin oils are believed to provide long lasting moisturized feel to the hair. Poly a-olefin oils having a molecular weight of less than Aabout 800 are useful herein. Such low molecular weight poly α-olefin oils are believed to provide a smooth, light, clean feel to the hair.

Citrate ester oils useful herein are those having a molecular weight of at least about 500 having the following formula:

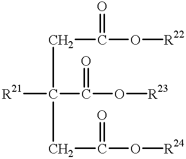

wherein R²¹ is OH or CH₃COO, and R²², R²³, and R²⁴, independently, are branched, straight, saturated, or unsaturated alkyl, aryl, and alkylaryl groups having from 1 to about 30 carbons. Preferably, R²¹ is OH, and R²², R²³, and R²⁴, independently, are branched, straight, saturated, or unsaturated alkyl, aryl, and alkylaryl groups having from 8 to about 22 carbons. More preferably, R²′, R²², R²³ and R²⁴ are defined so that the molecular weight of the compound is at least about 800.

Glyceryl ester oils useful herein are those having a molecular weight of at least about 500 and having the following formula:

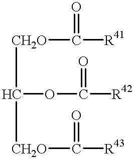

wherein R⁴¹, R⁴², and R⁴³, independently, are branched, straight, saturated, or unsaturated alkyl, aryl, and alkylaryl groups having from 1 to about 30 carbons. Preferably, R⁴¹, R⁴², and R⁴³, independently, are branched, straight, saturated, or unsaturated alkyl, aryl, and alkylaryl groups having from 8 to about 22 carbons. More preferably, R⁴¹, R⁴², and R⁴³ are defined so that the molecular weight of the compound is at least about 800.

Particularly useful pentaerythritol ester oils and trimethylol ester oils herein include pentaerythritol tetraisostearate, pentaerythritol tetraoleate, trimethylolpropane triisostearate, trimethylolpropane trioleate, and mixtures thereof. Such compounds are available from Kokyo Alcohol with tradenames KAKPTI™, KAKTTI™, and Shinnihon Rika with tradenames PTO™, ENUJERUBU TP3SO™.

Particularly useful poly α-olefin oils herein include polydecenes with tradenames PURESYN 6™ having a number average molecular weight of about 500 and PURESYN 100™ having a number average molecular weight of about 3000 and PURESYN 300™ having a number average molecular weight of about 6000 available from Mobil Chemical Co.

Particularly useful citrate ester oils herein include triisocetyl citrate with tradename CITMOL 316™ available from Bemel, triisostearyl citrate with tradename PELEMOL TISC™ available from Phoenix, and trioctyldodecyl citrate with tradename CITMOL 320™ available from Bemel.

Particularly useful glyceryl ester oils herein include triisostearin with tradename SUN ESPOL G-318™ available from Taiyo Kagaku, triolein with tradename CITHROL GTO™ available from Croda Surfactants Ltd., trilinolein with tradename EFADERMA-F™ available from Vevy, or tradename EFA-GLYCERIDES™ from Brooks.

Also suitable for use as conditioning agents in the inventive compositions described herein are polyol fatty acid polyesters. A “polyol” is a polyhydric alcohol containing at least 4, preferably from 4 to 11 hydroxyl groups. A “polyol fatty acid polyester” is a polyol having at least 4 fatty acid ester groups. Typically, at least about 85%, of the hydroxyl groups of the polyol are esterified. In the case of sucrose polyesters, typically from 7 to 8 of the hydroxyl groups of the polyol are esterified. The polyol fatty acid esters typically contain C₄ to C₂₆ fatty acid radicals. A preferred sucrose polyester for use herein is olestra, sold under the trade name OLEAN®, available from The Procter and Gamble Company. This oil, which is a blend of sucrose ester fatty acids (predominantly C₁₆ to C₁₈, and about 1% to about 2% C₁₄ to C₁₈), is described in U.S. Pat. Nos. 5,085,884, (Young, et al.) issued 4 February, 1992, and 5,422,131, (Elsen, et al.) issued Jun. 6, 1995, both of which descriptions are incorporated herein by reference.

Cationic Polymers

Cationic polymers are useful herein. As used herein, the term “polymer” shall include materials whether made by polymerization of one type of monomer or made by two (i e., copolymers) or more types of monomers.

Preferably, the cationic polymer is a water soluble cationic polymer. By “water soluble cationic polymer”, what is meant is a polymer which is sufficiently soluble in water to form a substantially clear solution to the naked eye at a concentration of 0.1% in water (distilled or equivalent) at 25° C. The preferred polymer will be sufficiently soluble to form a substantially clear solution at 0.5% concentration, more preferably at 1.0% concentration.

The cationic polymers hereof will generally have a weight average molecular weight which is at least about 5,000, typically at least about 10,000, and is less than about 10 million. Preferably, the molecular weight is from about 100,000 to about 2 million. The cationic polymers will generally have cationic nitrogen-containing moieties such as quaternary ammonium or cationic amino moieties, and mixtures thereof.

Any anionic counterions can be utilized for the cationic polymers so long as the water solubility criteria is met. Suitable counterions include halides (e.g., Cl, Br, I, or F, preferably Cl, Br, or I), sulfate, and methylsulfate. Others can also be used, as this list is not exclusive.

The cationic nitrogen-containing moiety will be present generally as a substituent, on a fraction of the total monomer units of the cationic hair conditioning polymers. Thus, the cationic polymer can comprise copolymers, terpolymers, etc. of quaternary ammonium or cationic amine-substituted monomer units and other non-cationic units referred to herein as spacer monomer units. Such polymers are known in the art, and a variety can be found in the CTFA Cosmetic Ingredient Dictionary, 3rd edition, edited by Estrin, Crosley, and Haynes, (The Cosmetic, Toiletry, and Fragrance Association, Inc., Washington, D.C., 1982).

The cationic amines can be primary, secondary, or tertiary amines, depending upon the particular species and the pH of the composition. In general, secondary and tertiary amines, especially tertiary amines, are preferred.

Amine-substituted vinyl monomers can be polymerized in the amine form, and then optionally can be converted to ammonium by a quaternization reaction. Amines can also be similarly quaternized subsequent to formation of the polymer. For example, tertiary amine functionalities can be quaternized by reaction with a salt of the formula R⁸⁸X wherein R⁸⁸ is a short chain alkyl, preferably a C₁-C₇ alkyl, more preferably a C₁-C₃ alkyl, and X is a salt forming anion as defined above.

Suitable cationic amino and quaternary ammonium monomers include, for example, vinyl compounds substituted with dialkylaminoalkyl acrylate, dialkylaminoalkyl methacrylate, monoalkylaminoalkyl acrylate, monoalkylaminoalkyl methacrylate, trialkyl methacryloxyalkyl ammonium salt, trialkyl acryloxyalkyl ammonium salt, diallyl quaternary ammonium salts, and vinyl quaternary ammonium monomers having cyclic cationic nitrogen-containing rings such as pyridinium, imidazolium, and quaternized pyrrolidone, e.g, alkyl vinyl imidazolium, alkyl vinyl pyridinium, alkyl vinyl pyrrolidone salts. The alkyl portions of these monomers are preferably lower allkyls such as the C₁-C₃ alkyls, more preferably C₁ and C₂ alkyls. Suitable amine-substituted vinyl monomers for use herein include dialkylaminoalkyl acrylate, dialkylaminoalkyl methacrylate, dialkylaminoalkyl acrylamide, and dialkylaminoalkyl methacrylamide, wherein the alkyl groups are preferably C₁-C₇ hydrocarbyls, more preferably C₁-C₃, alkyls.

The cationic polymers hereof can comprise mixtures of monomer units derived from amine- and/or quaternary ammonium-substituted monomer and/or compatible spacer monomers.

Suitable cationic hair conditioning polymers include, for example: copolymers of 1-vinyl-2-pyrrolidone and 1-vinyl-3-methylimidazolium salt (e.g., chloride salt) (referred to in the industry by the Cosmetic, Toiletry, and Fragrance Association, “CTFA”, as Polyquaternium-16), such as those commercially available from BASF Wyandotte Corp. (Parsippany, N.J., USA) under the LUVIQUAT tradename (e.g., LUVIQUAT FC 370™); copolymers of 1-vinyl-2-pyrrolidone and dimethylaminoethyl methacrylate (referred to in the industry by CTFA as Polyquaternium-11) such as those commercially available from Gaf Corporation (Wayne, N.J., USA) under the GAFQUAT tradename (e.g., GAFQUAT 755N™); cationic diallyl quaternary ammonium-containing polymers, including, for example, dimethyldiallylammonium chloride homopolymer and copolymers of acrylamide and dimethyldiallylammonium chloride, referred to in the industry (CTFA) as Polyquaternium 6 and Polyquaternium 7, respectively; and mineral acid salts of amino-alkyl esters of homo- and co-polymers of unsaturated carboxylic acids having from 3 to 5 carbon atoms, as described in U.S. Pat. No. 4,009,256.

Other cationic polymers that can be used include polysaccharide polymers, such as cationic cellulose derivatives and cationic starch derivatives.

Cationic polysaccharide polymer materials suitable for use herein include those of the formula:

[chemical structure]

wherein: Z⁷ is an anhydroglucose residual group, such as a starch or cellulose anhydroglucose residual, R⁸⁹ is an alkylene oxyalkylene, polyoxyalkylene, or hydroxyalkylene group, or combination thereof, R⁹⁰, R⁹¹, and R⁹² independently are alkyl, aryl, alkylaryl, arylalkyl, alkoxyalkyl, or alkoxyaryl groups, each group containing up to about 18 carbon atoms, and the total number of carbon atoms for each cationic moiety (i.e., the sum of carbon atoms in R⁹⁰, R⁹¹ and R⁹²) preferably being about 20 or less, and X is as previously described.

Cationic cellulose is available from Amerchol Corp. (Edison, N.J., USA) in their Polymer JR™ and LR™ series of polymers, as salts of hydroxyethyl cellulose reacted with trimethyl ammonium substituted epoxide, referred to in the industry (CTFA) as Polyquaternium 10. Another type of cationic cellulose includes the polymeric quaternary ammonium salts of hydroxyethyl cellulose reacted with lauryl dimethyl ammonium-substituted epoxide, referred to in the industry (CTFA) as Polyquaternium 24. These materials are available from Amerchol Corp. (Edison, N.J., USA) under the tradename Polymer LM-200™.

Other cationic polymers that can be used include cationic guar gum derivatives, such as guar hydroxypropyltrimonium chloride commercially available from Celanese Corp. in their Jaguar R series. Other materials include quaternary nitrogen-containing cellulose ethers as described in U.S. Pat. No. 3,962,418, and copolymers of etherified cellulose and starch as described in U.S. Pat. No. 3,958,581.

Particularly useful cationic polymers herein include Polyquaternium-7, Polyquaternium- 10, Polyquaternium-24, and mixtures thereof.

Additional Oily Compounds

Additional oily compounds useful herein include fatty alcohols and their derivatives, fatty acids and their derivatives, and hydrocarbons. The additional oily compounds useful herein may be volatile or nonvolatile, and have a melting point of not more than about 25° C. Without being bound by theory, it is believed that, the additional oily compounds may penetrate into the hair to modify the hydroxy bonds of the hair, thereby resulting in providing softness and flexibility to the hair. The additional oily compounds of this section are to be distinguished from the high melting point compounds described above. Nonlimiting examples of the additional oily compounds are found in International Cosmetic Ingredient Dictionary, Fifth Edition, 1993, and CTFA Cosmetic Ingredient Handbook, Second Edition, 1992.

The fatty alcohols useful herein include those having from about 10 to about 30 carbon atoms, preferably from about 12 to about 22 carbon atoms, and more preferably from about 16 to about 22 carbon atoms. These fatty alcohols can be straight or branched chain alcohols and can be saturated or unsaturated alcohols, preferably unsaturated alcohols. Nonlimiting examples of these compounds include oleyl alcohol, palmitoleic alcohol, isostearyl alcohol, isocetyl alcohol, undecanol, octyl dodecanol, octyl decanol, octyl alcohol, caprylic alcohol, decyl alcohol and lauryl alcohol.

The fatty acids useful herein include those having from about 10 to about 30 carbon atoms, preferably from about 12 to about 22 carbon atoms, and more preferably from about 16 to about 22 carbon atoms. These fatty acids can be straight or branched chain acids and can be saturated or unsaturated. Suitable fatty acids include, for example, oleic acid, linoleic acid, isostearic acid, linoleic acid, ethyl linoleic acid, ethyl linoleic acid, arachidonic acid, and ricinolic acid.

The fatty acid derivatives and fatty alcohol derivatives are defined herein to include, for example, esters of fatty alcohols, alkoxylated fatty alcohols, alkyl ethers of fatty alcohols, alkyl ethers of alkoxylated fatty alcohols, and bulky ester oils such as pentaerythritol ester oils, trimethylol ester oils, citrate ester oils, glyceryl ester oils, and mixtures thereof. Nonlimiting examples of fatty acid derivatives and fatty alcohol derivatives, include, for example, methyl linoleate, ethyl linoleate, isopropyl linoleate, isodecyl oleate, isopropyl oleate, ethyl oleate, octyldodecyl oleate, oleyl oleate, decyl oleate, butyl oleate, methyl oleate, octyldodecyl stearate, octyldodecyl isostearate, octyldodecyl isopalmitate, octyl isopelargonate, octyl pelargonate, hexyl isostearate, isopropyl isostearate, isodecyl isononanoate, isopropyl stearate, ethyl stearate, methyl stearate and Oleth-2. Bulky ester oils such as pentaerythritol ester oils, trimethylol ester oils, citrate ester oils and glyceryl ester oils useful herein are those which have a molecular weight of less than about 800, preferably less than about 500.

The hydrocarbons useful herein include straight chain, cyclic, and branched chain hydrocarbons which can be either saturated or unsaturated, so long as they have a melting point of not more than about 25° C. These hydrocarbons have from about 12 to about 40 carbon atoms, preferably from about 12 to about 30 carbon atoms, and preferably from about 12 to about 22 carbon atoms. Also encompassed herein are polymeric hydrocarbons of alkenyl monomers, such as polymers of C₂-₆ alkenyl monomers. These polymers can be straight or branched chain polymers. The straight chain polymers will typically be relatively short in length, having a total number of carbon atoms as described above. The branched chain polymers can have substantially higher chain lengths. The number average molecular weight of such materials can vary widely, but will typically be up to about 500, preferably from about 200 to about 400, and more preferably from about 300 to about 350. Also useful herein are the various grades of mineral oils. Mineral oils are liquid mixtures of hydrocarbons that are obtained from petroleum. Specific examples of suitable hydrocarbon materials include paraffin oil, mineral oil, dodecane, isododecane, hexadecane, isohexadecane, eicosene, isoeicosene, tridecane, tetradecane, polybutene, polyisobutene, and mixtures thereof. Preferred for use herein are hydrocarbons selected from the group consisting of mineral oil, poly α-olefin oils such as isododecane, isohexadecane, polybutene, polyisobutene, and mixtures thereof.

Commercially available fatty alcohols and their derivatives useful herein include: oleyl alcohol with tradename UNJECOL 9OBHR™ available from Shin Nihon Rika, various liquid esters with tradenames SCHERCEMOL™ series available from Scher, and hexyl isostearate with a tradename HIS™ and isopropyl isostearate having a tradename ZPIS™ available from Kokyu Alcohol. Commercially available bulky ester oils useful herein include: trimethylolpropane tricaprylate/tricaprate with tradename MOBIL ESTER P43™ from Mobil Chemical Co. Commercially available hydrocarbons useful herein include isododecane, isohexadecane, and isoeicosene with tradenames PERMETHYL 99A™, PERMETHYL 101A™, and PERMETHYL 182™, available from Presperse (South Plainfield N.J., USA), a copolymer of isobutene and normal butene with tradenames INDOPOL H-100™ available from Amoco Chemicals (Chicago Ill., and USA), mineral oil with tradename BENOL™ available from Witco, isoparaffin with tradename ISOPAR™ from Exxon Chemical Co. (Houston Tex., USA.)

Other Optional Components

The compositions of the present invention may also contain vitamins and amino acids such as: water soluble vitamins such as vitamin B1, B2, B6, B12, C, pantothenic acid, pantothenyl ethyl ether, panthenol, biotin, and their derivatives, water soluble amino acids such as asparagine, alanin, indole, glutamic acid and their salts, water insoluble vitamins such as vitamin A, D, E, and their derivatives, water insoluble amino acids such as tyrosine, tryptamine, and their salts.

The compositions of the present invention may also contain pigment materials such as inorganic, nitroso, monoazo, disazo, carotenoid, triphenyl methane, triaryl methane, xanthene, quinoline, oxazine, azine, anthraquinone, indigoid, thionindigoid, quinacridone, phthalocianine, botanical, natural colors, including: water soluble components such as those having C. I. Names: Acid Red 18, 26, 27,33, 51, 52, 87, 88, 92, 94, 95, Acid Yellow 1, 3, 11, 23, 36, 40, 73, Food Yellow 3, Food Green 3, Food blue 2, Food Red 1, 6, Acid Blue 5, 9, 74, Pigment Red 57-1, 53(Na), Basic Violet 10, Solvent Red 49, Acid orange 7, 20, 24, Acid Green 1, 3, 5, 25, Solvent Green 7, Acid Violet 9, 43; water insoluble components such as those having C. I. Names: Pigment Red 53(Ba), 49(Na), 49(Ca), 49(Ba), 49(Sr), 57, Solvent Red 23, 24, 43, 48, 72, 73, Solvent Orange 2, 7, Pigment Red 4, 24, 48, 63(Ca)3, 64, Vat Red 1, Vat blue 1, 6, Pigment Orange 1, 5, 13, Solvent Yellow 5, 6, 33, Pigment Yellow 1, 12, Solvent Green 3, Solvent Violet 13, Solvent Blue 63, Pigment Blue 15, titanium dioxides, chlorophyllin copper complex, ultramarines, aluminum powder, bentonite, calcium carbonate, barium sulfate, bismuthine, calcium sulfate, carbon black, bone black, chromic acid, cobalt blue, gold, ferric oxides, hydrated ferric oxide, ferric ferrocyanide, magnesium carbonate, manganous phosphate, silver, and zinc oxides.

The compositions of the present invention may also contain antimicrobial agents which are useful as cosmetic biocides and antidandruff agents including: water soluble components such as piroctone olamine, water insoluble components such as 3,4,4′-trichlorocarbanilide (trichlosan), triclocarban and zinc pyrithione.

The compositions of the present invention may also contain chelating agents such as: 2,2′-dipyridylamine; 1,10-phenanthroline {o-phenanthroline}; di-2-pyridyl ketone; 2,3-bis(2-pyridyl) pyrazine; 2,3-bis(2-pyridyl)-5,6-dihydropyrazine; 1,1′-carbonyldiimidazole; 2,4-bis(5,6-diphenyl-1,2,4-triazine-3-yl)pyridine; 2,4,6-tri(2-pyridyl)-1,3,5-triazine; 4,4′-dimethyl-2,2′dipyridyl; 2,2′-biquinoline; di-2-pyridyl glyoxal {2,2′-pyridil}; 2-(2-pyridyl)benzimidazole; 2,2′-bipyrazine; 3-(2-pyridyl)5,6-diphenyl-1,2,4-trazine; 3-(4-phenyl-2-pyridyl)-5-phenyl-1,2,4-triazine; 3-(4-phenyl-2-pyridyl)-5,6-diphenyl-1,2,4-triazine; 2,3,5,6-tetrakis-(2′-pyridyl)-pyrazine; 2,6-pyridinedi-carboxylic acid; 2,4,5-trihydroxypyrimidine; phenyl 2-pyridyl ketoxime; 3-amino-5,6-dimethyl- 1,2,4-triazine; 6-hydroxy-2-phenyl-3(2H)-pyridazinone; 2,4-pteridinediol {lumazine}; 2,2′-dipyridyl; and 2,3-dihydroxypyridine.

Visible Particle

The compositions of the present invention may further contain a visible particle. By definition, a “visible particle” is a particle which can be distinctively detected as an individual particle by the naked eye when comprised in the present composition, and which is stable in the present composition. The visible particle can be of any size, shape, or color, according to the desired characteristic of the product, so long as it is distinctively detected as an individual particle by the naked eye. Generally, the visible particle has an average diameter of from about 50 μm to about 3000 μm, preferably from about 100 μm to about 1000 μm, more preferably from about 300 μm to about 1000 μm. By stable, it is meant that the visible particles are not disintegrated, agglomerated, or separated under normal shelf conditions. In one preferred embodiment of the present invention, the composition is substantially transparent. In such an embodiment, the visible particles provide a highly suitable aesthetic benefit. What is generally meant by transparent, is that a black substance having the size of a 1 cm×1 cm square can be detected by the naked eye through 1 cm thickness of the present composition.

The visible particles herein are used at levels of from about 0.01% to about 5% by weight of the composition.

The visible particle herein comprises a structural material and preferably an encompassed material.

The structural material provides a certain strength to the visible particle so that they retain their distinctively detectable structure in the present composition under normal shelf conditions. In one preferred embodiment, the structural material further can be broken and disintegrated with very little shear on the hand with the fingers upon use.

Visible particles useful herein include capsules, shelled particles, beads, pellets, droplets, pills, caplets, tablets, grains, flakes, powders and granules. The visible particles can be solid or liquid, filled or un-filled, so long as they are stable in the present composition. The structural material used for making the visible particles varies depending on the compatibility with other components, as well as material, if any, to be encompassed in the visible particles. Exemplary materials for making the visible particles herein include: polysaccharide and saccharide derivatives such as crystalline cellulose, cellulose acetate, cellulose acetate butyrate, cellulose acetate phthalate, cellulose nitrate, ethylcellulose, hydroxypropylcellulose, hydroxypropylmethylcellulose, hydroxypropyl-methylcellulose phthalate, methyl cellulose, sodium carboxymethylcellulose, gum acacia (gum arabic), agar, agarose, maltodextrin, sodium alginate, calcium alginate, dextran, starch, galactose, glucosamine, cyclodextrin, chitin, amylose, amylopectin, glycogen, laminaran, lichenan, curdlan, inulin, levan, pectin, mannan, xylan, alginic acid, arabic acid, glucommannan, agarose, agaropectin, prophyran, carrageenen, fiucoidan, glycosaminoglycan, hyaluronic acid, chondroitin, peptidoglycan, lipopolysaccharide, guar gum, starch, and starch derivatives; oligosaccharides such as sucrose, lactose, maltose, uronic acid, muramic acid, cellobiose, isomaltose, planteose, melezitose, gentianose, maltotriose, stachyose, glucoside and polyglucoside; monosaccharides such as glucose, fructose, and mannose; synthetic polymers such as acrylic polymers and copolymers including polyacrylamide, poly(alkyl cyanoacrylate), and poly(ethylene-vinyl acetate), and carboxyvinyl polymer, polyamide, poly(methyl vinyl ether-maleic anhydride), poly(adipyl-L-lysine), polycarbonate, polyterephthalamide, polyvinyl acetate phthalate, poly-(terephthaloyl-L-lysine), polyarylsulfone, poly(methylmethacrylate), poly(ε-caprolactone), polyvinylpyrrolidone, polydimethylsiloxane, polyoxyethylene, polyester, polyglycolic acid, polylactic acid, polyglutamic acid, polylysine, polystyrene, poly(styrene-acrylonitrile), polyimide, and poly(vinyl alcohol); and other material such as fat, fatty acid, fatty alcohol, milk solids, molasses, gelatin, gluten, albumin, shellac, caseinate, bees wax, carnauba wax, spermaceti wax, hydrogenated tallow, glycerol monopalmitate, glycerol dipalmitate, hydrogenated castor oil, glycerol monostearate, glycerol distearate, glycerol tristearate, 12-hydroxystearyl alcohol, protein, and protein derivatives; and mixtures thereof. Components herein may be described in other sections as useful components for the present composition. The components herein, however, are substantially used to make the structure of the visible particles, and are not dissolved or dispersed in the bulk of the present composition under normal shelf conditions.

Highly preferable structural material herein comprises components selected from the group consisting of polysaccharides and their derivatives, saccharides and their derivatives, oligosaccharides, monosaccharides, and mixtures thereof, still preferably, components from the above mentioned group wherein components having various water solubility are selected. In a particularly preferred embodiment, the structural material is made of components selected from the group consisting of cellulose, cellulose derivatives, saccharides, and mixtures thereof.

The visible particle herein may encompass, contain, or be filled with an encompassed material. Such encompassed material can be water soluble or water insoluble, and comprise components such as: vitamins, amino acids, proteins and protein derivatives, herbal extracts, pigments, dyes, antimicrobial agents, chelating agents, UV absorbers, optical brighteners, silicone compounds, perfumes, humectants which are generally water soluble, additional conditioning agents which are generally water insoluble, and mixtures thereof. In one embodiment, water soluble components are preferred encompassed material. In another embodiment, components selected from the group consisting of vitamins, amino acids, proteins, protein derivatives, herbal extracts, and mixtures thereof are preferred encompassed material. In yet another embodiment, components selected from the group consisting of vitamin E, panthenyl ethyl ether, panthenol, Polygonum multiflori extracts, and mixtures thereof are preferred encompassed material.

Vitamins and amino acids useful as encompassed material herein include: water soluble vitamins such as vitamin B1, B2, B6, B12, C, pantothenic acid, panthenyl ethyl ether, panthenol, biotin, and their derivatives, water soluble amino acids such as asparagine, alanin, indole, glutamic acid and their salts, water insoluble vitamins such as vitamin A, D, E, and their derivatives, water insoluble amino acids such as tyrosine, tryptamine, and their salts.

Pigments useful as encompassed material herein include inorganic, nitroso, monoazo, bisazo, carotenoid, triphenyl methane, triaryl methane, xanthene, quinoline, oxazine, azine, anthraquinone, indigoid, thionindigoid, quinacridone, phthalocianine, botanical, natural colors, including: water soluble components such as those having C. I. Names: Acid Red 18, 26, 27,33, 51, 52, 87, 88, 92, 94, 95, Acid Yellow 1, 3, 11, 23, 36, 40, 73, Food Yellow 3, Food Green 3, Food blue 2, Food Red 1, 6, Acid Blue 5, 9, 74, Pigment Red 57-1, 53(Na), Basic Violet 10, Solvent Red 49, Acid orange 7, 20, 24, Acid Green 1, 3, 5, 25, Solvent Green 7, Acid Violet 9, 43; water insoluble components such as those having C. I. Names: Pigment Red 53(Ba), 49(Na), 49(Ca), 49(Ba), 49(Sr), 57, Solvent Red 23, 24, 43, 48, 72, 73, Solvent Orange 2, 7, Pigment Red 4, 24, 48, 63(Ca)3, 64, Vat Red 1, Vat blue 1, 6, Pigment Orange 1, 5, 13, Solvent Yellow 5, 6, 33, Pigment Yellow 1, 12, Solvent Green 3, Solvent Violet 13, Solvent Blue 63, Pigment Blue 15, titanium dioxides, chlorophyllin copper complex, ultramarines, aluminum powder, bentonite, calcium carbonate, barium sulfate, bismuthine, calcium sulfate, carbon black, bone black, chromic acid, cobalt blue, gold, ferric oxides, hydrated ferric oxide, ferric ferrocyanide, magnesium carbonate, manganous phosphate, silver, and zinc oxides.

Antimicrobial agents useful as encompassed material include those useful as cosmetic biocides and antidandruff agents including: water soluble components such as piroctone olamine, water insoluble components such as 3,4,4′-trichlorocarbanilide (trichlosan), trichlocarban and zinc pyrithione.

Chelating agents useful as encompassed material include: 2,2′-dipyridylamine; 1,10-phenanthroline {o-phenanthroline}; di-2-pyridyl ketone; 2,3-bis(2-pyridyl) pyrazine; 2,3-bis(2-pyridyl)-5,6-dihydropyrazine; 1,1′-carbonyldiimidazole; 2,4-bis(5,6-diphenyl-1,2,4-triazine-3-yl)pyridine; 2,4,6-tri(2-pyridyl)-1,3,5-triazine; 4,4′-dimethyl-2,2′dipyridyl; 2,2′-biquinoline; di-2-pyridyl glyoxal {2,2′-pyridil}; 2-(2-pyridyl)benzimidazole; 2,2′-bipyrazine; 3-(2-pyridyl)5,6-diphenyl-1,2,4-trazine; 3-(4-phenyl-2-pyridyl)-5-phenyl-1,2,4-triazine; 3-(4-phenyl-2-pyridyl)-5,6-diphenyl-1,2,4-triazine; 2,3,5,6-tetralkis-(2′-pyridyl)-pyrazine; 2,6-pyridinedicarboxylic acid; 2,4,5-trihydroxypyrimidine; phenyl 2-pyridyl ketoxime; 3-amino-5 ,6-dimethyl- 1,2,4-triazine; 6-hydroxy-2-phenyl-3(2H)-pyridazinone; 2,4-pteridinediol {lumazine}; 2,2-′dipyridyl; and 2,3-dihydroxypyridine.

Useful silicone compounds, humectants, additional conditioning agents, UV absorbers, optical brighteners, and herbal extracts for encompassed material are the same as those exemplified in other portions of the specification. The components herein, however, are substantially retained within the breakable visible particles, and are substantially not dissolved in the bulk of the present composition under normal shelf conditions.

Particularly useful commercially available visible particles herein are those with tradenames Unisphere™ and Unicerin™ available from Induchem AG (Switzerland), and Confetti Dermal Essentials available from United-Guardian Inc. (N.Y., USA). Unisphere™ and Unicerin™ particles are made of microcrystalline cellulose, hydroxypropyl cellulose, lactose, vitamins, pigments, and proteins. Upon use, the Unisphere™ and Unicerin™ particles can be disintegrated with very little shear on the hand with the fingers with practically no resistance, and readily dissolve in the composition.

The compositions of the present invention may include other additional components, which may be selected by the artisan according to the desired characteristics of the final product and which are suitable for rendering the composition more cosmetically or aesthetically acceptable or to provide them with additional usage benefits. Such other additional components generally are used individually at levels of from about 0.001% to about 10%, preferably up to about 5% by weight of the composition.

A wide variety of other additional components can be formulated into the present compositions. These include: other conditioning agents such as hydrolyzed collagen with tradename Peptein 2000™ available from Hormel, vitamin E with tradename Emix-d™ available from Eisai, panthenol available from Roche, panthenyl ethyl ether available from Roche, hydrolyzed keratin, proteins, plant extracts, and nutrients; hair-fixative polymers such as amphoteric fixative polymers, cationic fixative polymers, anionic fixative polymers, nonionic fixative polymers, and silicone grafted copolymers; preservatives such as benzyl alcohol, methyl paraben, propyl paraben and imidazolidinyl urea; pH adjusting agents, such as citric acid, sodium citrate, succinic acid, phosphoric acid, sodium hydroxide, sodium carbonate; salts, in general, such as potassium acetate and sodium chloride; coloring agents, such as any of the FD&C or D&C dyes; hair oxidizing (bleaching) agents, such as hydrogen peroxide, perborate and persulfate salts; hair reducing agents such as the thioglycolates; perfumes; and sequestering agents, such as disodium ethylenediamine tetra-acetate; ultraviolet and infrared screening and absorbing agents such as octyl salicylate, antidandruff agents such as zinc pyrithione; and mixtures thereof.

### PRODUCT FORMS

The hair conditioning compositions of the present invention can be transparent or opaque, and can be formulated in a wide variety of product forms, including but not limited to creams, gels, emulsions, and mousses.

The compositions of the present invention are preferably transparent. What is meant by transparent, is that a black substance having the size of a 1 cm×1 cm square can be detected by the naked eye through 1 cm thickness of the present composition.

### METHOD OF USE

The hair cosmetic compositions of the present invention are used in conventional ways to provide the volumizing, conditioning, styling and other benefits of the present invention. Such method of use depends upon the type of composition employed but generally involves providing hair, or a hair sample, having a bulk hair area, applying an effective amount of the product to the hair, and then drying the hair. Before drying, the composition will be allowed to remain on the hair (as in the case of gels, lotions, and creams). “Effective amount” means an amount sufficient enough to provide the desired increase in bulk hair volume and the conditioning benefits. In general, from about 1 g to about 50 g is applied to the hair, and/or the scalp. The phrase “increase in bulk hair volume” as used herein is not equal to fly-away hair. Fly-away hair is due to the increased level of static, and represents volume increase of only very minor amount of the hair as a whole, and is not desirable. On the other hand, increase in bulk hair volume as used herein relates to the volume increase of the hair as a whole while controlling fly-away of the hair.

During the applying step, the hair care composition may be distributed throughout the hair, typically by rubbing or massaging the hair and scalp, or the composition may be selectively applied to certain parts of the hair. The composition is preferably applied to wet or damp hair prior to drying of the hair. After such hair care compositions are applied to the hair, the hair is dried and styled in accordance with the preference of the user. In the alternative, it may be applied to already dry hair, and the hair is then combed or styled, and dried in accordance with the preference of the user.

### EXAMPLES

The following examples further describe and demonstrate embodiments within the scope of the present invention. The examples are given solely for the purpose of illustration and are not to be construed as limitations of the present invention, as many variations thereof are possible without departing from the spirit and scope of the invention. Ingredients are identified by chemical or CTFA name, or otherwise defined below.

All percentages herein are based upon the total weight of the compositions, and all such weight percentages as they pertain to listed ingredients are based on the active level and, therefore, do not include carriers or by-products that may be included in commercially available materials.

I. Following are non-limiting examples of conditioning formulations:

Compositions (wt %)

| | Ex. 1 | Ex. 2 | Ex.3 | Ex. 4 | Ex. 5 | Ex. 6 | Ex. 7 | Ex. 8 | Ex. 9 |
|---|---|---|---|---|---|---|---|---|---|
| Acrylic acid alkyl acrylate copolymer 1 | 0.35 | 0.35 | — | 0.5 | — | 0.5 | 0.35 | — | — |
| *1 | | | | | | | | | |
| Acrylic acid alkyl acrylate copolymer 2 | — | — | 0.3 | — | 0.5 | — | — | — | — |
| *2 | | | | | | | | | |
| Polyvinylpyrrolidone/Vinyl acetate | — | — | 0.8 | — | — | 1.0 | 1.0 | — | — |
| copolymer 1 *3 | | | | | | | | | |
| Polyvinylpyrrolidone/Vinyl acetate | — | — | — | 1.0 | — | 0.5 | — | — | — |
| copolymer 2 *4 | | | | | | | | | |
| Polyvinylpyrrolidone/Vinyl acetate | — | — | — | — | 1.0 | — | — | — | — |
| copolymer 3 *5 | | | | | | | | | |
| Triethanolamine *6 | 0.3 | 0.3 | 0.5 | 0.6 | 0.7 | 0.6 | 0.5 | — | — |
| Cetyl Alcohol *7 | — | — | 0.8 | — | 0.2 | — | — | — | — |
| Stearyl Alcohol *8 | — | — | — | — | — | 0.5 | — | — | — |
| Behenyl Alcohol *9 | — | — | — | 0.8 | — | — | — | — | — |
| Dimethicone/Dimethiconol *10 | 1.0 | 1.0 | 0.5 | 1.0 | — | — | 1.0 | 1.0 | 1.0 |
| Cyclomethicone/Dimethiconol *11 | — | — | — | — | — | 3.0 | — | — | — |
| Cyclomethicone/Dimethicone *12 | — | — | — | — | 3.0 | — | — | — | — |
| Cyclomethicone *13 | — | — | — | — | 2.0 | — | — | — | — |
| Polyquaternium-39 *14 | 0.2 | 0.2 | 1.0 | 0.1 | 0.1 | 0.1 | 0.2 | 0.1 | 0.1 |
| Polyquaternium-47 *15 | — | — | — | — | — | — | — | — | — |
| Carbomer 1 *16 | 0.1 | 0.1 | — | 0.1 | 0.2 | — | 0.1 | — | — |
| Carbomer 2 *17 | — | — | 0.3 | — | — | 0.5 | — | — | — |
| Acrylates/Steareth-20 Methacrylate | — | — | 0.2 | — | — | — | — | | |
| Copolymer *18 | | | | | | | | | |
| Propylene Glycol *19 | — | — | 2.0 | — | — | — | — | — | — |
| Hexylene Glycol *20 | — | — | — | — | — | — | — | — | — |
| Polyethylene Glycol 200 *21 | 2.0 | 2.0 | 1.0 | 4.0 | 2.0 | 4.0 | 2.0 | 2.0 | 2.0 |
| Polygonum multiflori extract *22 | — | — | — | 0.1 | — | — | 0.1 | — | — |
| Vitamin E *23 | — | — | — | 0.05 | — | — | — | — | — |
| Panthenol *24 | 0.1 | — | 0.1 | — | — | — | — | — | — |
| Benzophenone-4 *25 | 0.05 | 0.05 | 0.1 | — | 0.1 | | 0.05 | 0.1 | 0.1 |
| Octyl Methoxycinnamate *26 | — | — | | 0.1 | — | 0.2 | — | — | — |
| Microparticles 1 *27 | — | 1.5 | 1.25 | — | 0.5 | — | — | 2.0 | 1.0 |
| Microparticles 2 *28 | — | — | — | 0.2 | — | 0.5 | 1.0 | — | — |
| Microspheres 1 *29 | — | 1.25 | — | 1.0 | — | 1.0 | — | 1.0 | — |
| Microspheres 2 *30 | — | — | 0.75 | — | 1.0 | — | 1.0 | — | 1.0 |
| Methyl Paraben | 0.2 | 0.2 | 0.2 | 0.2 | 0.2 | 0.2 | 0.2 | 0.2 | 0.2 |
| Phenoxyethanol | 0.3 | 0.3 | 0.3 | 0.3 | 0.3 | 0.3 | 0.3 | 0.3 | 0.3 |
| Disodium EDTA | 0.13 | 0.13 | 0.13 | 0.1 | 0.1 | 0.1 | 0.1 | 0.1 | 0.1 |
| Perfume solution | 0.12 | 0.12 | 0.12 | 0.1 | 0.1 | 0.1 | 0.1 | 0.1 | 0.1 |
| Visible particles *31 | — | — | — | — | — | — | 0.1 | — | — |
| Visible particles *32 | — | — | — | — | — | — | 0.1 | — | — |
| Hydrophobically mod. cellulose ether | — | — | — | — | — | — | — | — | 0.5 |
| *33 | | | | | | | | | |
| Acrylates copolymer *34 | — | — | — | — | — | — | — | — | 0.3 |
| Crosslinked copolymer *35 | — | — | — | — | — | — | — | — | 0.2 |

| | |
|---|---|
| Deionized Water | q.s. to 100% |

Definitions of Components
*1 Acrylic acid alkyl acrylate copolymer 1: PEMULEN TR-1 ™ available from B.F. Goodrich
*2 Acrylic acid alkyl acrylate copolymer 2: PEMULEN TR-2 ™ available from B.F. Goodrich
*3 Polyvinylpyrrolidone/Vinyl acetate copolymer 1: Luviskol 73W ™ with a mole ratio of vinylpyrrolidone monomer to vinyl acetate monomer 7:3 available from BASF
*4 Polyvinylpyrrolidone/Vinyl acetate copolymer 2: Luviskol 64W ™ with a mole ratio of vinylpyrrolidone monomer to vinyl acetate monomer 6:4 available from BASF
*5 Polyvinylpyrrolidone/Vinyl acetate copolymer 3: Polyvinylpyrrolidone/Vinyl acetate/Vinyl propionate copolymer having tradename Luviskol VAP343E with a mole ratio of vinylpyrrolidone monomer to vinyl acetate monomer and vinyl propionate monomer 3:4:3 available from BASF
*6 Triethanolamine: Triethanolamine available from Nippon Shokubai
*7 Cetyl Alcohol: Konol series available from Shinnihon Rika
*8 Stearyl Alcohol: Konol series available from Shinnihon Rika
*9 Behenyl Alcohol: Behenyl alcohol 65, 80: available from Nikko Chemical
*10 Dimethicone/Dimethiconol: DC-1403 ™ available from Dow Corning
*11 Cyclomethicone/Dimethiconol: DCQ2-1401 ™ available from Dow Corning
*12 Cyclomethicone/Dimethicone: Gum/Cyclomethicone blend available from Shin-Etsu
*13 Cyclomethicone: DC345 ™ available from Dow Corning
*14 Polyquaternium-39: Merquat Plus 3330 ™ available from Calgon
*15 Polyquaternium-47: Merquat 2001 ™ available from Calgon
*16 Carbomer 1: Carbopol 981 ™ available from B.F. Goodrich
*17 Carbomer 2: Carbopol Ultrez 10 ™ available from B.F. Goodrich
*18 Acrylates/Steareth-20 Methacrylate Copolymer: Acrysol 22 ™ available from Rohm and Hass
*19 Propylene Glycol: Available from BASF
*20 Hexylene Glycol: Hexylene glycol available from Mitsui Toatsu
*21 Polyethylene Glycol 200: Carbowax PEG200 ™ available from Union Carbide
*22 Polygonum multiflori extract: Polygonum multiflori extract obtained from Occupational Medicine, CAPM.
*23 Vitamin E: Emix-d Available from Eisai
*24 Panthenol: Panthenol Available from Roche
*25 Benzophenone-4: Uvnul MS-40 ™ available from BASF
*26 Octyl Methoxycinnamate: Parasol MCX ™ available from Roche
*27 Microparticles 1: Bentone MA (available from Reox)
*28 Microparticles 2: Laponite XLS ™ (available from Southern Clay)
*29 Microspheres 1: 091 DE available as EXPANCEL ™, from Akzo Nobel
*30 Microspheres 2: 091 DE80 available as EXPANCEL ™, from Akzo Nobel
*31 Visible particles 1: Unispheres AGE-52 ™ available from Induchem
*32 Visible particles 2: Unispheres YE-501 ™ available from Induchem
*33 Hydrophobically modified cellulose ether: Natrosol Plus CS Grade 330 ™ available from Aqualon
*34 Acrylates copolymer 1: Structure plus available from National starch
*35 Crosslinked polymer: Polyquaternium 37 sold as Salcare 96 ™ available from Allied Colloids

Method of Preparation of Conditioner Formulations

The polymeric materials such as the carboxylic acid/alkyl carboxylate copolymer, polyvinylpyrrolidone/vinyl acetate copolymer, amphoteric conditioning polymer, and additional viscosity modifier, if present, are dispersed in water at room temperature, mixed by vigorous agitation, and heated to 50° C. The high melting point compounds, if included, are added to the mixture with agitation at above 70° C. by either melting such components or by dissolving such components. Then the neutralizing agent is added to the mixture.

After neutralizing, the mixture is cooled to below 40° C., and then the remaining components are added to the mixture with agitation.

The above examples have many advantages, such as improved conditioning benefits to the hair such as smoothness, softness, and reduction of friction, while providing increase in bulk hair volume. These compositions are also easy to apply on the hair, and they leave the hair and hands with a clean feeling.

It is understood that the examples and embodiments described herein are for illustrative purposes only and that various modifications or changes in light thereof will be suggested to one skilled in the art without departing from its spirit and scope.

II. The following are non-limiting examples of mousse formulations:

### Example 10:

A. In a sanitized 4L beaker, 2835.80 g of water is added and heated to 125° F+/− 5° F. With mechanical stirring at about 400 rpm, 90.00 g of Polyquaternium-4, 3.00 g of Disodium EDTA, and 0.12 g of Citric Acid is added to the water. Ingredients are mixed for 15 minutes or until completely dissolved. Solution is stopped from being heated and 7.50 g of C9-11 Pareth 8 and 15.00 g of Propylene Glycol is added to the solution, with continuous stirring. When the batch temperature is under 110° F., 21.22 g of DMDM Hydantoin, 1.20 g of Panthenol, 2.70 g of Panthyl Ethyl Ether, 2.4 g of Keratin amino acids, 0.15 g of Myristyol Hydrolyzed Collagen and 4.50 g of perfume are added. Mechanical stirring is ended and 16.41 g of Laponite XLS™ microparticles are manually stirred into the solution. pH of the solution is measured and adjusted by adding Citric Acid, with a target of 5.85+/−0.45. Mousse product is produced by filling cans with 171.13 g of this solution and 16.1 g of Propellant A-46.

B. The procedure of embodiment A is repeated except that 16.41 g of EXPANCEL 551 DE™ microspheres are added by manually sturring. The microspheres and Laponite microparticles may be added sequentially, in any order, or simultaneously.

III. The following are non-limiting examples of gel formulations:

### Example 11:

A. Premix A is made by placing 2977.50 g of water into a 4L sanitized beaker. The water is heated to and maintained at 70-80° F. for remainder of the batch. Agitation is begun at a maximum safe speed and 22.50 g of Carbomer 940 is slowly sifted in. Solution is mixed until completely dissolved, for at least 30 minutes.

Premix B is made by placing 341.16 g of water into a 2L sanitized beaker and heating it to 125° F.+/−5° F. With mechanical stirring, 4.14 g of Tetrasodium EDTA, 12.60 g of Polyquaternium-4, and 18.00 g of Isosteareth-20 are added. Solution is stopped from being heated and 63.00 g of Polyquaternium-11, 144.00 g of PVPNVA Copolymer, 0.72 g of Panthenol, 0.18 g of Octyl Salicylate and 0.18 g of Vitamin E Acetate are added. When batch temperature is under 100° F., 2.00 g of perfume and 13.32 g of DMDM Hydantoin are added. Solution is mixed until homogenous.

Gel formulation is made by placing 2400.00 g of Premix A into a sanitized 4L beaker. Stirring by hand, 15.00 g of Laponite XLS™ and 11.76 g of Triethanolamine are added to Premix A. Then, 500.10 g of Premix B is added and solution is stirred by hand. With continued manual agitation, 3.54 g of Triethanolamine is added dropwise. Solution is mixed until homogeneous for at least 15 minutes. pH of the solution is measured and adjusted, if necessary, by adding Triethanolamine, with a target of 5.90+/−0.30.

B. The procedure of embodiment B is repeated except that 15.00 g of EXPANCEL 551 DE 50™ microspheres are also added to Premix A either before, concurrently with or after additions of the Laponite XLS™.

### Test Method for Measuring Bulk Volume Differences in Hair

Specimens of the Examples are tested for their volume effects on hair utilizing the following procedure.

4 gram/8 inch fine hair switches (round) are used; at least 3 switches are used per treatment.

1. All hair switches used in this method are first cleaned by taking them through two Prell shampoo cycles. Excess water is squeezed from the switches after rinsing.

2. Working with each switch individually, 0.075 cc Conditioner Prototype/gram of hair (3 g conditioner per 4 g switch) is applied to the damp hair. The conditioner is worked into the hair for 30 seconds. The switch is combed to ensure no tangles and the hair is smoothed flat between fingers (all switches should have the same approximate shape after this step). Treated switches are then hung on a drying rack.

3. Once all switches have been treated, the drying rack is placed in a 75° F./50% relative humidity room for overnight air drying.

4. A picture is taken of the dry switches before dry combing.

5. Dry switches are combed, one at a time, using a fine tooth comb (5 strokes per switch).

6. A static gun is used on the hanging switches to remove static.

7. A picture is taken immediately. The picture is used, in addition to visual inspection, to identify any differences in bulk volume between treated switches.

8. Each switch is rated in the following manner:

A control is included with every run of this method. The control is the leave-in conditioner product with no volume-up additives.

Differences in bulk volume are noted between switches treated with the control product and those treated with the volume-up prototypes. Bulk volume differences existing both before and after dry combing are noted.

If the control is considered to have a bulk volume rating of 1, then small bulk volume increases are given a value of 2 and significant increases in bulk volume are given a 3. Pictures of switches are especially useful for seeing differences in bulk volume between switches and are used for the actual quantification procedure. These supplement the qualitative visual observation of bulk volume differences between switches.

Results:

Hair switches treated with the formulations containing microparticles, or containing microparticles, and microspheres, are compared with the hair switches treated with a formulation containing no microparticles or microspheres. Significantly high volume, as evaluated by the procedure described above, is noticed for the hair switches treated with formulations containing microparticles, or containing microparticles and microspheres.

The specific embodiments and examples set forth herein are illustrative only and are not intended to limit the scope of the claims defining the invention. Additional embodiments and examples within the scope of the claims will be apparent in view of the present specification.

What is claimed is:

1. A leave-in hair cosmetic composition, comprising non-spherical microparticles exhibiting a mean particle size of less than about 100 μm in its longest dimension, a water-soluble or water-swellable polymer and aqueous carrier, wherein the combination of the polymer and the microparticles results in a film-forming network.

2. A leave-in hair cosmetic composition, comprising:
(i) from 0.25% to about 15%, by weight of the composition, of non-spherical microparticles which exhibit a mean particle size of less than about 100 μm in its longest dimension;
(ii) from about 0.025% to about 10%, by weight of the composition, of a water-soluble or swellable polymer; and
(iii) an aqueous carrier, wherein the combination of the polymer, the microparticles and the microspheres results in a film-forming network.

3. A leave-in hair cosmetic composition according to claim 1, wherein said microparticles are formed from a natural clay or processed or synthetic clay.

4. A leave-in hair cosmetic composition according to claim 3, wherein said microparticles are formed of a natural clay.

5. A leave-in hair cosmetic composition according to claim 4, wherein said natural clay is Bentone Mass.

6. A leave-in hair cosmetic composition according to claim 3, wherein said microparticles are formed of a synthetic clay.

7. A leave-in hair cosmetic composition according to claim 6, wherein said clay is Laponite XLS.

8. A leave-in hair cosmetic composition according to claim 1, wherein said microparticles exhibit a mean particle size ranging from about 1 μm to about 60 μm.

9. A leave-in hair cosmetic composition according to claim 1, wherein said microparticles exhibit a mean particle size ranging from about 1 μm to about 20 μm.

10. A leave-in hair cosmetic composition according to claim 1, wherein surfaces of said microparticles are modified with a charge, hydrophobic functional groups, hydrophilic functional groups, or a combination thereof.

11. A leave-in hair cosmetic composition according to claim 10, wherein the surfaces of said microparticles are ionically charged.

12. A leave-in hair cosmetic composition according to claim 10, wherein said functional groups comprise alkyl, hydroxy, alkoxy, amino, carboxy, sulfate or halide groups, or mixtures thereof.

13. A leave-in hair cosmetic composition according to claim 1, further comprising fluid encapsulated, flexible microspheres exhibiting a mean particle size of less than about 300 μm in diameter.

14. A leave-in hair cosmetic composition according to claim 2, further comprising from about 0.25% to about 15%, by weight of the composition, of fluid encapsulated flexible microspheres which exhibit a mean particle size of less than about 300 μm in diameter.

15. A leave-in hair cosmetic composition according to claim 13, wherein said microspheres have a density of from about 5 kg/m³ to about 200 kg/m³.

16. A leave-in hair cosmetic composition according to claim 13, wherein said microspheres have a density of from about 5 kg/m³ to about 100 kg/m³.

17. A leave-in hair cosmetic composition according to claim 13, wherein said microspheres comprise a thermoplastic material wall.

18. A leave-in hair cosmetic composition according to claim 17, wherein said thermoplastic material is a polymer or copolymer of at least one monomer selected from the group consisting of acrylates, methacrylates, styrene, substituted styrene, unsaturated dihalides, acrylonitriles, and methacrylonitriles.

19. A leave-in hair cosmetic composition according to claim 17, wherein said thermoplastic material is a polymer or copolymer comprising amide, ester, urethane, urea, ether, carbonate, acetal, sulfide, phosphate, phosphonate ester, or siloxane linkages.

20. A leave-in hair cosmetic composition according to claim 18, wherein said thermoplastic material is a polymer or copolymer of at least one monomer selected from the group consisting of acrylates, styrene, vinylidene chloride, acrylonitriles, and methacrylonitriles.

21. A leave-in hair cosmetic composition according to claim 20, wherein said thermoplastic material is a copolymer of acrylonitrile and methacrylonitrile.

22. A leave-in hair cosmetic composition according to claim 13, wherein said microspheres exhibit a mean particle size ranging from about 5 μm to about 100 μm.

23. A leave-in hair cosmetic composition according to claim 13, wherein said microspheres exhibit a mean particle size ranging from about 5 μm to about 80 μm.

24. A leave-in hair cosmetic composition according to claim 13, wherein surfaces of said microspheres are modified by attachment of an ionic group.

25. A leave-in hair cosmetic composition according to claim 13, wherein the surfaces of said microspheres are modified by attachment of an organic or inorganic material.

26. A leave-in hair cosmetic composition according to claim 1, wherein the aqueous carrier is selected from the group consisting of a leave-in conditioning product, a leave-in styling product, a leave-in coloring product, and mixtures thereof.

27. A leave-in hair conditioning composition comprising:
(i) from about 0.01% to about 10%, by weight of the composition, of a carboxylic acid/carboxylate copolymer;
(ii) from about 0.25% to about 10%, by weight of the composition, of non-spherical microparticles which exhibit a mean particle size of less than about 100 μm in its longest dimension; and
(iii) an aqueous carrier, wherein the combination of the copolymer and the microparticles results in a film-forming network.

28. A leave-in hair conditioning composition according to claim 27, further comprising from about 0.25% to about 10%, by weight of the composition, of fluid-encapsulated, flexible microspheres which exhibit a mean particle size of less than about 300 μm in diameter.

29. A leave-in hair conditioning composition comprising:
(1) a thickening system comprising at least 2 thickening agents selected from (i), (ii) and (iii):
(i) a hydrophobically modified cellulose ether;
(ii) an acrylate copolymer comprising by weight:
(a) from about 5% to about 80% of an acrylate monomer selected from the group consisting of a C₁-C₆ alkyl ester of acrylic acid, a C₁-C₆ alkyl ester of methacrylic acid, and mixtures thereof;
(b) from about 5% to about 80% of a monomer selected from the group consisting of a vinyl-substituted heterocyclic compound containing at least one of a nitrogen or sulfur atom, a (meth)acrylamide, a mono- or di-(C₁-C₄)alkylamino(C₁-C₄)alkyl-(meth)acrylate, a mono- or di-(C₁-C₄)alkylamino(C₁-C₄)alkyl(meth)-acrylamide, and mixtures thereof; and
(c) from 0% to about 30% of an associative monomer;
(iii) a crosslinked polymer having the formula (A)ₘ(B)ₙ(C)ₚ, wherein:
(A) is selected from the group consisting of a dialkylaminoalkyl acrylate, a quaternized dialkylaminoalkyl acrylate, an acid addition salt of a quaternized dialkylaminoalkyl acrylate, and mixtures thereof;
(B) is selected from the group consisting of a dialkylaminoalkyl methacrylate, a quaternized dialkylaminoalkyl methacrylate, an acid addition salt of a quaternized dialkylaminoalkyl methacrylate, and mixtures thereof,
(C) is a nonionic monomer polymerizable with (A) or (B); and m, n, and p are independently zero or greater, but at least one of m or n is one or greater;
(2) from about 0.25% to about 10%, by weight of the composition, of non-spherical microparticles which exhibit a mean particle size of less than about 100 μm in its longest dimension; and
(3) an aqueous carrier, wherein the combination of the copolymer and the microparticles results in a solid continuous or semi-continuous film network.

30. A leave-in hair conditioning composition according to claim 29, further comprising from about 0.25% to about 1I%, by weight of the composition, of fluid-encapsulated, flexible microspheres which exhibit a mean particle size of less than about 300 μm in diameter.

31. A method for enhancing hair volume by applying to hair an effective amount of a composition according to claim 1.

32. A method for enhancing hair volume by applying to hair an effective amount of a composition according to claim 13.

33. A method for enhancing hair volume by applying to hair an effective amount of a composition according to claim 27.

34. A method for enhancing hair volume by applying to hair an effective amount of a composition according to claim 29.
